(19) United States
(12) Patent Application Publication
(10) Pub. No.: US 2012/0157494 A1
(43) Pub. Date: Jun. 21, 2012

(54) ISOINDOLYL COMPOUNDS

(76) Inventors: Ralph New Harris, III, Redwood City, CA (US); David Bruce Repke, Milpitas, CA (US); Keith A.M. Walker, Los Altos Hills, CA (US)

(21) Appl. No.: 13/325,113

(22) Filed: Dec. 14, 2011

Related U.S. Application Data
(60) Provisional application No. 61/423,633, filed on Dec. 16, 2010.

(51) Int. Cl. A61K 31/4545; A61K 31/4035; C07D 401/14; C07D 401/10; C07D 209/46; A61P 11/06; C07D 405/14; C07D 409/14; C07D 417/14; A61P 19/02; A61P 29/00; A61P 15/00; A61P 41/00; A61P 25/00; A61P 35/00; A61P 31/04; A61P 33/00; A61P 31/12; A61P 1/00; A61P 11/00; A61P 11/08; A61K 31/454

(52) U.S. Cl. 514/318; 514/323; 514/416; 546/194; 546/201; 548/472

(57) ABSTRACT

Compounds of the formula I:

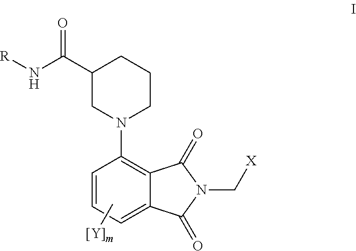

or pharmaceutically acceptable salts thereof, wherein the variables are as defined herein. Also disclosed are methods of making the compounds and using the compounds for treatment of diseases associated with the P2X7 purinergic receptor.

### PRIORITY TO RELATED APPLICATIONS

This application is entitled to the benefit of U.S. provisional patent application Ser. No. 61/423,633 filed on Dec. 16, 2010, the disclosure of which is incorporated herein by reference.

### FIELD OF THE INVENTION

This invention pertains to compounds useful for treatment of diseases associated with P2X purinergic receptors, and more particularly to P2X₇ modulators usable for treatment of autoimmune and inflammatory diseases.

### BACKGROUND OF THE INVENTION

P2X purinergic receptors are ATP-activated ionotropic receptors having seven subtypes. The P2X7 receptor subtype (also known as the P2Z receptor) is a ligand-gated ion channel found on mast cells, peripheral macrophages, lymphocytes, erythrocytes, fibroblasts and epidermal langerhans cells. Activation of P2X7 receptor on such immune system cells results in release of interleukin-1beta. The P2X7 receptor is also found on microglia, Schwann cells and astrocytes within the central nervous system.

Antagonists of P2X7 have been shown to block P2×7-mediated IL-1beta release and P2×7-mediated cation flux. Mice lacking the P2X7 receptor show a lack of inflammatory and neuropathic hypersensitivity to mechanical and thermal stimuli. P2X7 is thus believed to have a role in inflammatory responses.

Modulators of the P2X7 receptor thus may have utility in the treatment of disease states such as rheumatoid arthritis, osteoarthritis, psoriasis, allergic dermatitis, asthma, chronic obstructive pulmonary disease, airways hyper-responsiveness, septic shock, glomerulonephritis, irritable bowel disease, diabetes and Crohn's disease. P2X7 modulators may also be useful for treatment of pain, including chronic pain, neuropathic pain, and pain associated inflammatory processes and degenerative conditions.

There is accordingly a need for compounds that act as modulators of P2X receptors, including antagonists of P2X7 receptor, as well as a need for methods of treating diseases, conditions and disorders mediated by P2X7 The present invention satisfies these needs as well as others.

### SUMMARY OF THE INVENTION

The application provides a compound of formula I:

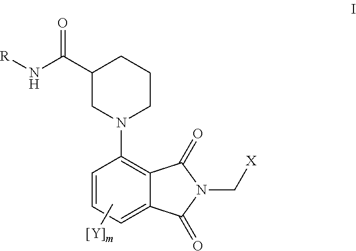

wherein:

### R is R′ or CH₂R′;

R′ is aryl, cycloalkyl, heterocycloalkyl, heteroaryl, optionally substituted with one or more R¹;

R¹ is halo, hydroxy, lower alkyl, lower haloalkyl, lower hydroxyalkyl, lower alkoxy, lower thioalkyl, lower alkyl sulfonyl, or lower haloalkoxy;

X is lower alkyl, aryl, cycloalkyl, heterocycloalkyl, or heteroaryl, optionally substituted with one or more X¹;

X¹ is halo, hydroxy, lower alkyl, lower haloalkyl, lower hydroxyalkyl, or lower alkoxy;

Y is halo;
m is 0, 1, or 2;
or a pharmaceutically acceptable salt thereof.

The application provides a compound of formula II:

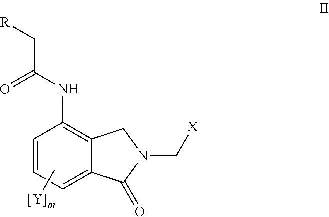

wherein:
R is aryl or cycloalkyl, optionally substituted with one or more R¹;

R¹ is halo, hydroxy, lower alkyl, lower haloalkyl, lower hydroxyalkyl, lower alkoxy, lower thioalkyl, lower alkyl sulfonyl, or lower haloalkoxy;

X is lower alkyl, optionally substituted with one or more X¹;

X¹ is halo, hydroxy, lower alkyl, lower haloalkyl, lower hydroxyalkyl, or lower alkoxy;

Y is halo;
m is 0, 1, or 2;
or a pharmaceutically acceptable salt thereof.

The application provides a pharmaceutical composition comprising:
(a) a pharmaceutically acceptable carrier; and
(b) a compound of Formula I or Formula II.

The application provides a method for treating arthritis, said method comprising administering to a subject in need thereof a therapeutically effective amount of a compound of Formula I or Formula II.

### DETAILED DESCRIPTION OF THE INVENTION

### Definitions

Unless otherwise stated, the following terms used in this Application, including the specification and claims, have the definitions given below. It must be noted that, as used in the specification and the appended claims, the singular forms “a”, “an,” and “the” include plural referents unless the context clearly dictates otherwise.

“Alkyl” means the monovalent linear or branched saturated hydrocarbon moiety, consisting solely of carbon and hydrogen atoms, having from one to twelve carbon atoms. “Lower alkyl” refers to an alkyl group of one to six carbon atoms, i.e. C₁-C₆alkyl. Examples of alkyl groups include, but are not limited to, methyl, ethyl, propyl, isopropyl, isobutyl, sec-butyl, tert-butyl, pentyl, n-hexyl, octyl, dodecyl, and the like.

“Alkenyl” means a linear monovalent hydrocarbon radical of two to six carbon atoms or a branched monovalent hydrocarbon radical of three to six carbon atoms, containing at least one double bond, e.g., ethenyl, propenyl, and the like.

“Alkynyl” means a linear monovalent hydrocarbon radical of two to six carbon atoms or a branched monovalent hydrocarbon radical of three to six carbon atoms, containing at least one triple bond, e.g., ethynyl, propynyl, and the like.

“Alkylene” means a linear saturated divalent hydrocarbon radical of one to six carbon atoms or a branched saturated divalent hydrocarbon radical of three to six carbon atoms, e.g., methylene, ethylene, 2,2-dimethylethylene, propylene, 2-methylpropylene, butylene, pentylene, and the like.

“Alkoxy” and “alkyloxy”, which may be used interchangeably, mean a moiety of the formula —OR, wherein R is an alkyl moiety as defined herein. Examples of alkoxy moieties include, but are not limited to, methoxy, ethoxy, isopropoxy, and the like.

“Alkoxyalkyl” means a moiety of the formula R^{a}—O—R^{b}—, where R^{a} is alkyl and R^{b} is alkylene as defined herein. Exemplary alkoxyalkyl groups include, by way of example, 2-methoxyethyl, 3-methoxypropyl, 1-methyl-2-methoxyethyl, 1-(2-methoxyethyl)-3-methoxypropyl, and 1-(2-methoxyethyl)-3-methoxypropyl.

“Alkoxyalkoxy” means a group of the formula —O—R—R′ wherein R is alkylene and R′ is alkoxy as defined herein.

“Alkylcarbonyl” means a moiety of the formula —C(O)—R, wherein R is alkyl as defined herein.

“Alkoxycarbonyl” means a group of the formula —C(O)—R wherein R is alkoxy as defined herein.

“Alkylcarbonylalkyl” means a group of the formula —R—C(O)—R wherein R is alkylene and R′ is alkyl as defined herein.

“Alkoxycarbonylalkyl” means a group of the formula —R—C(O)—R wherein R is alkylene and R′ is alkoxy as defined herein.

“Alkoxycarbonylalkoxy” means a group of the formula —O—R—C(O)—R′ wherein R is alkylene and R′ is alkoxy as defined herein.

“Hydroxycarbonylalkoxy” means a group of the formula —O—R—C(O)—OH wherein R is alkylene as defined herein.

“Alkylaminocarbonylalkoxy” means a group of the formula —O—R—C(O)—NHR′ wherein R is alkylene and R′ is alkyl as defined herein.

“Dialkylaminocarbonylalkoxy” means a group of the formula —O—R—C(O)—NR′R″ wherein R is alkylene and R′ and R″ are alkyl as defined herein.

“Alkylaminoalkoxy” means a group of the formula —O—R—NHR′ wherein R is alkylene and R′ is alkyl as defined herein.

“Dialkylaminoalkoxy” means a group of the formula —O—R—NR′R′ wherein R is alkylene and R′ and R″ are alkyl as defined herein.

“Alkylsulfonyl” means a moiety of the formula —SO₂—R, wherein R is alkyl as defined herein.

“Alkylsulfonylalkyl” means a moiety of the formula —R′—SO₂—R″ where R′ is alkylene and R″ is alkyl as defined herein.

“Alkylsulfonylalkoxy” means a group of the formula —O—R—SO₂—R′ wherein R is alkylene and R′ is alkyl as defined herein.

“Amino” means a moiety of the formula —NRR′ wherein R and R′ each independently is hydrogen or alkyl as defined herein. “Amino” thus includes “alkylamino (where one of R and R′ is alkyl and the other is hydrogen) and “dialkylamino (where R and R′ are both alkyl.

“Aminocarbonyl” means a group of the formula —C(O)—R wherein R is amino as defined herein.

“Alkoxyamino” means a moiety of the formula —NR—OR′ wherein R is hydrogen or alkyl and R′ is alkyl as defined herein.

“Alkylsulfanyl” means a moiety of the formula —SR wherein R is alkyl as defined herein.

“Aminoalkyl” means a group —R—R′ wherein R′ is amino and R is alkylene as defined herein. “Aminoalkyl” includes aminomethyl, aminoethyl, 1-aminopropyl, 2-aminopropyl, and the like. The amino moiety of “aminoalkyl” may be substituted once or twice with alkyl to provide “alkylaminoalkyl” and “dialkylaminoalkyl” respectively. “Alkylaminoalkyl” includes methylaminomethyl, methylaminoethyl, methylaminopropyl, ethylaminoethyl and the like.

“Dialkylaminoalkyl” includes dimethylaminomethyl, dimethylaminoethyl, dimethylaminopropyl, N-methyl-N-ethylaminoethyl, and the like.

“Aminoalkoxy” means a group —OR—R′ wherein R′ is amino and R is alkylene as defined herein.

“Alkylsulfonylamido” means a moiety of the formula —NR′SO₂—R wherein R is alkyl and R′ is hydrogen or alkyl.

“Aminocarbonyloxyalkyl” or “carbamylalkyl” means a group of the formula —R—O—C(O)—NR′R″ wherein R is alkylene and R′, R″ each independently is hydrogen or alkyl as defined herein.

“Alkynylalkoxy” means a group of the formula —O—R—R′ wherein R is alkylene and R′ is alkynyl as defined herein.

“Aryl” means a monovalent cyclic aromatic hydrocarbon moiety consisting of a mono-, bi- or tricyclic aromatic ring. The aryl group can be optionally substituted as defined herein. Examples of aryl moieties include, but are not limited to, phenyl, naphthyl, phenanthryl, fluorenyl, indenyl, pentalenyl, azulenyl, oxydiphenyl, biphenyl, methylenediphenyl, aminodiphenyl, diphenylsulfidyl, diphenylsulfonyl, diphenylisopropylidenyl, benzodioxanyl, benzofuranyl, benzodioxylyl, benzopyranyl, benzoxazinyl, benzoxazinonyl, benzopiperadinyl, benzopiperazinyl, benzopyrrolidinyl, benzomorpholinyl, methylenedioxyphenyl, ethylenedioxyphenyl, and the like, including partially hydrogenated derivatives thereof, each being optionally substituted.

“Arylalkyl” and “Aralkyl”, which may be used interchangeably, mean a radical-R^{a}R^{b} where R^{a} is an alkylene group and R^{b} is an aryl group as defined herein; e.g., phenylalkyls such as benzyl, phenylethyl, 3-(3-chlorophenyl)-2-methylpentyl, and the like are examples of arylalkyl.

“Arylsulfonyl” means a group of the formula —SO₂—R wherein R is aryl as defined herein.

“Aryloxy” means a group of the formula —O—R wherein R is aryl as defined herein.

“Aralkyloxy” means a group of the formula —O—R—R″ wherein R is alkylene and R′ is aryl as defined herein.

“Carboxy” or “hydroxycarbonyl”, which may be used interchangeably, means a group of the formula —C(O)—OH.

“Cyanoalkyl” means a moiety of the formula —R′—R″, where R′ is alkylene as defined herein and R″ is cyano or nitrile.

“Cycloalkyl” means a monovalent saturated carbocyclic moiety consisting of mono- or bicyclic rings. Preferred cycloalkyl are unsubstituted or substituted with alkyl. Cycloalkyl can optionally be substituted with one or more substituents, wherein each substituent is independently hydroxy, alkyl, alkoxy, halo, haloalkyl, amino, monoalkylamino, or dialkylamino, unless otherwise specifically indicated. Examples of cycloalkyl moieties include, but are not limited to, cyclopropyl, cyclobutyl, cyclopentyl, cyclohexyl, cycloheptyl, and the like, including partially unsaturated (cycloalkenyl) derivatives thereof.

“Cycloalkylalkyl” means a moiety of the formula —R′—R″, where R′ is alkylene and R″ is cycloalkyl as defined herein.

“Cycloalkylalkoxy” means a group of the formula —O—R—R′ wherein R is alkylene and R′ is cycloalkyl as defined herein.

“Heteroalkyl” means an alkyl radical as defined herein wherein one, two or three hydrogen atoms have been replaced with a substituent independently selected from the group consisting of —OR^{a}, —NR^{b}R^{c} and —S(O)—R^{d} (where n is an integer from 0 to 2), with the understanding that the point of attachment of the heteroalkyl radical is through a carbon atom, wherein R^{a} is hydrogen, acyl, alkyl, cycloalkyl, or cycloalkylalkyl; R^{b} and R^{c} are independently of each other hydrogen, acyl, alkyl, cycloalkyl, or cycloalkylalkyl; and when n is 0, R^{d} is hydrogen, alkyl, cycloalkyl, or cycloalkylalkyl, and when n is 1 or 2, R^{d} is alkyl, cycloalkyl, cycloalkylalkyl, amino, acylamino, monoalkylamino, or dialkylamino. Representative examples include, but are not limited to, 2-hydroxyethyl, 3-hydroxypropyl, 2-hydroxy-1-hydroxymethylethyl, 2,3-dihydroxypropyl, 1-hydroxymethylethyl, 3-hydroxybutyl, 2,3-dihydroxybutyl, 2-hydroxy-1-methylpropyl, 2-aminoethyl, 3-aminopropyl, 2-methylsulfonylethyl, aminosulfonylmethyl, aminosulfonylethyl, aminosulfonylpropyl, methylaminosulfonylmethyl, methylaminosulfonylethyl, methylaminosulfonylpropyl, and the like.

“Heteroaryl” means a monocyclic or bicyclic radical of 5 to 12 ring atoms having at least one aromatic ring containing one, two, or three ring heteroatoms selected from N, O, or S, the remaining ring atoms being C, with the understanding that the attachment point of the heteroaryl radical will be on an aromatic ring. The heteroaryl ring may be optionally substituted as defined herein. Examples of heteroaryl moieties include, but are not limited to, optionally substituted imidazolyl, oxazolyl, isoxazolyl, thiazolyl, isothiazolyl, oxadiazolyl, thiadiazolyl, pyrazinyl, thienyl, benzothienyl, thiophenyl, furanyl, pyranyl, pyridyl, pyrrolyl, pyrazolyl, pyrimidyl, quinolinyl, isoquinolinyl, benzofuryl, benzothiophenyl, benzothiopyranyl, benzimidazolyl, benzooxazolyl, benzooxadiazolyl, benzothiazolyl, benzothiadiazolyl, benzopyranyl, indolyl, isoindolyl, triazolyl, triazinyl, quinoxalinyl, purinyl, quinazolinyl, quinolizinyl, naphthyridinyl, pteridinyl, carbazolyl, azepinyl, diazepinyl, acridinyl and the like, including partially hydrogenated derivatives thereof, each optionally substituted.

“Heteroarylalkyl” or “heteroaralkyl” means a group of the formula —R—R′ wherein R is alkylene and R′ is heteroaryl as defined herein.

“Heteroarylsulfonyl” means a group of the formula —SO₂—R wherein R is heteroaryl as defined herein.

“Heteroaryloxy” means a group of the formula —O—R wherein R is heteroaryl as defined herein.

“Heteroaralkyloxy” means a group of the formula —O—R—R″ wherein R is alkylene and R′ is heteroaryl as defined herein.

The terms “halo”, “halogen” and “halide”, which may be used interchangeably, refer to a substituent fluoro, chloro, bromo, or iodo.

“Haloalkyl” means alkyl as defined herein in which one or more hydrogen has been replaced with same or different halogen. Exemplary haloalkyls include —CH₂Cl, —CH₂CF₃, —CH₂CCl₃, perfluoroalkyl (e.g., —CF₃), and the like.

“Haloalkoxy” means a moiety of the formula —OR, wherein R is a haloalkyl moiety as defined herein. An exemplary haloalkoxy is difluoromethoxy.

“Heterocycloamino” means a saturated ring wherein at least one ring atom is N, NH or N-alkyl and the remaining ring atoms form an alkylene group.

“Heterocyclyl” means a monovalent saturated moiety, consisting of one to three rings, incorporating one, two, or three or four heteroatoms (chosen from nitrogen, oxygen or sulfur). The heterocyclyl ring may be optionally substituted as defined herein. Examples of heterocyclyl moieties include, but are not limited to, optionally substituted piperidinyl, piperazinyl, homopiperazinyl, azepinyl, pyrrolidinyl, pyrazolidinyl, imidazolinyl, imidazolidinyl, pyridinyl, pyridazinyl, pyrimidinyl, oxazolidinyl, isoxazolidinyl, morpholinyl, thiazolidinyl, isothiazolidinyl, quinuclidinyl, quinolinyl, isoquinolinyl, benzimidazolyl, thiadiazolylidinyl, benzothiazolidinyl, benzoazolylidinyl, dihydrofuryl, tetrahydrofuryl, dihydropyranyl, tetrahydropyranyl, thiamorpholinyl, thiamorpholinylsulfoxide, thiamorpholinylsulfone, dihydroquinolinyl, dihydrisoquinolinyl, tetrahydroquinolinyl, tetrahydrisoquinolinyl, and the like.

“Heterocyclylalkyl” means a moiety of the formula —R—R′ wherein R is alkylene and R′ is heterocyclyl as defined herein.

“Heterocyclyloxy” means a moiety of the formula —OR wherein R is heterocyclyl as defined herein.

“Heterocyclylalkoxy” means a moiety of the formula —OR—R′ wherein R is alkylene and

R′ is heterocyclyl as defined herein.

“Hydroxyalkoxy” means a moiety of the formula —OR wherein R is hydroxyalkyl as defined herein. Exemplary hydroxyalkoxy include, for example, 3-methoxy-2-hydroxy-propyl and 3-hydroxy-2-methoxy-propyl.

“Hydroxyalkoxyalkyl” means a moiety of the formula —ROR′ wherein R is alkylene and R′ is hydroxyalkyl as defined herein, and R or R′ or both are substituted with hydroxy.

“Hydroxyalkylamino” means a moiety of the formula —NR—R′ wherein R is hydrogen or alkyl and R′ is hydroxyalkyl as defined herein.

“Hydroxyalkylaminoalkyl” means a moiety of the formula —R—NR′—R″ wherein R is alkylene, R′ is hydrogen or alkyl, and R″ is hydroxyalkyl as defined herein.

“Hydroxycarbonylalkyl” or “carboxyalkyl” means a group of the formula —R—(CO)—OH where R is alkylene as defined herein.

“Hydroxycarbonylalkoxy” means a group of the formula —O—R—C(O)—OH wherein R is alkylene as defined herein.

“Hydroxyalkyloxycarbonylalkyl” or “hydroxyalkoxycarbonylalkyl” means a group of the formula —R—C(O)—O—R—OH wherein each R is alkylene and may be the same or different.

“Hydroxyalkyl” means an alkyl moiety as defined herein, substituted with one or more, preferably one, two or three hydroxy groups, provided that the same carbon atom does not carry more than one hydroxy group. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 2-hydroxy-1-hydroxymethylethyl, 2,3-dihydroxybutyl, 3,4-dihydroxybutyl and 2-(hydroxymethyl)-3-hydroxypropyl.

“Hydroxycycloalkyl” means a cycloalkyl moiety as defined herein wherein one, two or three hydrogen atoms in the cycloalkyl radical have been replaced with a hydroxy substituent. Representative examples include, but are not limited to, 2-, 3-, or 4-hydroxycyclohexyl, and the like.

“Alkoxy hydroxyalkyl” and “hydroxy alkoxyalkyl”, which may be used interchangeably, means an alkyl as defined herein that is substituted at least once with hydroxy and at least once with alkoxy. “Alkoxy hydroxyalkyl” and “hydroxy alkoxyalkyl” thus encompass, for example, 2-hydroxy-3-methoxy-propan-1-yl and the like.

“Urea” or “ureido” means a group of the formula —NR′—C(O)—NR″R′″ wherein R′, R″ and R′″ each independently is hydrogen or alkyl.

“Carbamate” means a group of the formula —O—C(O)—NR′R″ wherein R′ and R″ each independently is hydrogen or alkyl.

“Carboxy” means a group of the formula —O—C(O)—OH.

“Sulfonamido” means a group of the formula —SO₂—NR′R″ wherein R′, R″ and R′″ each independently is hydrogen or alkyl.

“Optionally substituted”, when used in association with “aryl”, “phenyl”, “heteroaryl”, “cycloalkyl” or “heterocyclyl”, means an aryl, phenyl, heteroaryl, cycloalkyl or heterocyclyl which is optionally substituted independently with one to four substituents, preferably one or two substituents selected from alkyl, cycloalkyl, cycloalkylalkyl, heteroalkyl, hydroxyalkyl, halo, nitro, cyano, hydroxy, alkoxy, amino, acylamino, mono-alkylamino, di-alkylamino, haloalkyl, haloalkoxy, heteroalkyl, —COR, —SO₂R (where R is hydrogen, alkyl, phenyl or phenylalkyl), —(CR′R″)ₙ—COOR (where n is an integer from 0 to 5, R′ and R″ are independently hydrogen or alkyl, and R is hydrogen, alkyl, cycloalkyl, cycloalkylalkyl, phenyl or phenylalkyl), or —(CR′R″)ₙ—CONR^{a}R^{b} (where n is an integer from 0 to 5, R′ and R″ are independently hydrogen or alkyl, and R^{a} and R^{b} are, independently of each other, hydrogen, alkyl, cycloalkyl, cycloalkylalkyl, phenyl or phenylalkyl). Certain preferred optional substituents for “aryl”, “phenyl”, “heteroaryl”, “cycloalkyl” or “heterocyclyl” include alkyl, halo, haloalkyl, alkoxy, cyano, amino and alkylsulfonyl. More preferred substituents are methyl, fluoro, chloro, trifluoromethyl, methoxy, amino and methanesulfonyl.

“Leaving group” means the group with the meaning conventionally associated with it in synthetic organic chemistry, i.e., an atom or group displaceable under substitution reaction conditions. Examples of leaving groups include, but are not limited to, halogen, alkane- or arylenesulfonyloxy, such as methanesulfonyloxy, ethanesulfonyloxy, thiomethyl, benzenesulfonyloxy, tosyloxy, and thienyloxy, dihalophosphinoyloxy, optionally substituted benzyloxy, isopropyloxy, acyloxy, and the like.

“Modulator” means a molecule that interacts with a target. The interactions include, but are not limited to, agonist, antagonist, and the like, as defined herein.

“Optional” or “optionally” means that the subsequently described event or circumstance may but need not occur, and that the description includes instances where the event or circumstance occurs and instances in which it does not.

“Disease” and “Disease state” means any disease, condition, symptom, disorder or indication.

“Inert organic solvent” or “inert solvent” means the solvent is inert under the conditions of the reaction being described in conjunction therewith, including for example, benzene, toluene, acetonitrile, tetrahydrofuran, N,N-dimethylformamide, chloroform, methylene chloride or dichloromethane, dichloroethane, diethyl ether, ethyl acetate, acetone, methyl ethyl ketone, methanol, ethanol, propanol, isopropanol, tert-butanol, dioxane, pyridine, and the like. Unless specified to the contrary, the solvents used in the reactions of the present invention are inert solvents.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

“Pharmaceutically acceptable salts” of a compound means salts that are pharmaceutically acceptable, as defined herein, and that possess the desired pharmacological activity of the parent compound. Such salts include:

acid addition salts formed with inorganic acids such as hydrochloric acid, hydrobromic acid, sulfuric acid, nitric acid, phosphoric acid, and the like; or formed with organic acids such as acetic acid, benzenesulfonic acid, benzoic, camphorsulfonic acid, citric acid, ethanesulfonic acid, fumaric acid, glucoheptonic acid, gluconic acid, glutamic acid, glycolic acid, hydroxynaphtoic acid, 2-hydroxyethanesulfonic acid, lactic acid, maleic acid, malic acid, malonic acid, mandelic acid, methanesulfonic acid, muconic acid, 2-naphthalenesulfonic acid, propionic acid, salicylic acid, succinic acid, tartaric acid, p-toluenesulfonic acid, trimethylacetic acid, and the like; or

salts formed when an acidic proton present in the parent compound either is replaced by a metal ion, e.g., an alkali metal ion, an alkaline earth ion, or an aluminum ion; or coordinates with an organic or inorganic base. Acceptable organic bases include diethanolamine, ethanolamine, N-methylglucamine, triethanolamine, tromethamine, and the like. Acceptable inorganic bases include aluminum hydroxide, calcium hydroxide, potassium hydroxide, sodium carbonate and sodium hydroxide.

The preferred pharmaceutically acceptable salts are the salts formed from acetic acid, hydrochloric acid, sulphuric acid, methanesulfonic acid, maleic acid, phosphoric acid, tartaric acid, citric acid, sodium, potassium, calcium, zinc, and magnesium.

It should be understood that all references to pharmaceutically acceptable salts include solvent addition forms (solvates) or crystal forms (polymorphs) as defined herein, of the same acid addition salt.

“Protective group” or “protecting group” means the group which selectively blocks one reactive site in a multifunctional compound such that a chemical reaction can be carried out selectively at another unprotected reactive site in the meaning conventionally associated with it in synthetic chemistry. Certain processes of this invention rely upon the protective groups to block reactive nitrogen and/or oxygen atoms present in the reactants. For example, the terms “amino-protecting group” and “nitrogen protecting group” are used interchangeably herein and refer to those organic groups intended to protect the nitrogen atom against undesirable reactions during synthetic procedures. Exemplary nitrogen protecting groups include, but are not limited to, trifluoroacetyl, acetamido, benzyl (Bn), benzyloxycarbonyl (carbobenzyloxy, CBZ), p-methoxybenzyloxycarbonyl, p-nitrobenzyloxycarbonyl, tert-butoxycarbonyl (BOC), and the like. The artisan in the art will know how to choose a group for the ease of removal and for the ability to withstand the following reactions.

“Solvates” means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

“Subject” means mammals and non-mammals. Mammals means any member of the mammalia class including, but not limited to, humans; non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex.

“Arthritis” means diseases or conditions damage to joints of the body and pain associated with such joint damage. Arthritis includes rheumatoid arthritis, osteoarthritis, psoriatic arthritis, septic arthritis and gouty arthritis.

“Pain” includes, without limitation, inflammatory pain; surgical pain; visceral pain; dental pain; premenstrual pain; central pain; pain due to burns; migraine or cluster headaches; nerve injury; neuritis; neuralgias; poisoning; ischemic injury; interstitial cystitis; cancer pain; viral, parasitic or bacterial infection; post-traumatic injury; or pain associated with irritable bowel syndrome.

“Therapeutically effective amount” means an amount of a compound that, when administered to a subject for treating a disease state, is sufficient to effect such treatment for the disease state. The “therapeutically effective amount” will vary depending on the compound, disease state being treated, the severity or the disease treated, the age and relative health of the subject, the route and form of administration, the judgment of the attending medical or veterinary practitioner, and other factors.

The terms “those defined above” and “those defined herein” when referring to a variable incorporates by reference the broad definition of the variable as well as preferred, more preferred and most preferred definitions, if any.

“Treating” or “treatment” of a disease state includes:
(i) preventing the disease state, i.e. causing the clinical symptoms of the disease state not to develop in a subject that may be exposed to or predisposed to the disease state, but does not yet experience or display symptoms of the disease state.
(ii) inhibiting the disease state, i.e., arresting the development of the disease state or its clinical symptoms, or
(iii) relieving the disease state, i.e., causing temporary or permanent regression of the disease state or its clinical symptoms.

The terms “treating”, “contacting” and “reacting” when referring to a chemical reaction means adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

### Inhibitors of P2X7

The application provides a compound of formula I⁰:

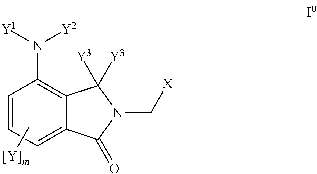

wherein:

### R is R′ or CH₂R′;

R′ is aryl, cycloalkyl, heterocycloalkyl, heteroaryl, optionally substituted with one or more R¹;

R¹ is halo, hydroxy, lower alkyl, lower haloalkyl, lower hydroxyalkyl, lower alkoxy, lower thioalkyl, lower alkyl sulfonyl, or lower haloalkoxy;

X is lower alkyl, aryl, cycloalkyl, heterocycloalkyl, or heteroaryl, optionally substituted with one or more X¹;

X¹ is halo, hydroxy, lower alkyl, lower haloalkyl, lower hydroxyalkyl, or lower alkoxy;

Y is halo;

### Y¹ is H;

### Y² is C(═O)CH₂R;

or Y¹ and Y² together form piperidine substituted with C(═O)NHR;

each Y³ is H;

or both Y³ together form oxo;

m is 0, 1, or 2;
or a pharmaceutically acceptable salt thereof.

The application provides a compound of formula I:

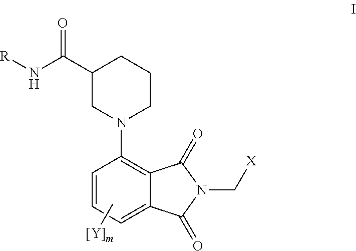

wherein:

### R is R′ or CH₂R′;

R′ is aryl, cycloalkyl, heterocycloalkyl, heteroaryl, optionally substituted with one or more R¹;

R¹ is halo, hydroxy, lower alkyl, lower haloalkyl, lower hydroxyalkyl, lower alkoxy, lower thioalkyl, lower alkyl sulfonyl, or lower haloalkoxy;

X is lower alkyl, aryl, cycloalkyl, heterocycloalkyl, or heteroaryl, optionally substituted with one or more X¹;

X¹ is halo, hydroxy, lower alkyl, lower haloalkyl, lower hydroxyalkyl, or lower alkoxy;

Y is halo;
m is 0, 1, or 2;
or a pharmaceutically acceptable salt thereof.

The application provides a compound of formula I, wherein X is pyridyl, optionally substituted with one or more X¹.

The application provides a compound of formula I, wherein R is phenyl, optionally substituted with one or more R¹.

The application provides a compound of formula I, wherein X is pyridyl, optionally substituted with one or more X¹ and R is phenyl, optionally substituted with one or more R¹.

The application provides a compound of formula I, wherein m is 0.

The application provides a compound of formula I, wherein m is 1.

The application provides a compound of formula I, wherein Y is Cl.

The application provides a compound of formula I, wherein m is 2. The application provides a compound of formula I, wherein m is 0, X is pyridyl, optionally substituted with one or more X¹ and R is phenyl, optionally substituted with one or more R¹.

The application provides a compound of formula I, wherein m is 1, X is pyridyl, optionally substituted with one or more X¹ and R is phenyl, optionally substituted with one or more R¹.

The application provides a compound of formula I, wherein m is 2, each Y is Cl, X is pyridyl, optionally substituted with one or more X¹ and R is phenyl, optionally substituted with one or more R¹.

The application provides a compound of formula I, wherein R¹ is halo.

The application provides a compound of formula I, wherein R¹ is alkoxy.

The application provides a compound of formula I, wherein R¹ is haloalkyl.

The application provides a compound of formula I, wherein R¹ is halo, X is pyridyl, optionally substituted with one or more X¹ and R is phenyl, optionally substituted with one or more R¹.

The application provides a compound of formula I, wherein R¹ is alkoxy, X is pyridyl, optionally substituted with one or more X¹ and R is phenyl, optionally substituted with one or more R¹.

The application provides a compound of formula I, wherein R¹ is haloalkyl, X is pyridyl, optionally substituted with one or more X¹ and R is phenyl, optionally substituted with one or more R¹.

The application provides a compound of formula I, wherein R is heterocycloalkyl, optionally substituted with one or more R¹.

The application provides a compound of formula I, wherein R is heteroaryl, optionally substituted with one or more R¹.

The application provides a compound of formula I, wherein R is cycloalkyl, optionally substituted with one or more R¹.

The application provides a compound of formula I, wherein X is heteroaryl, optionally substituted with one or more X¹.

The application provides a compound of formula I, wherein X is lower alkyl, optionally substituted with one or more X¹.

The application provides a compound of formula II:

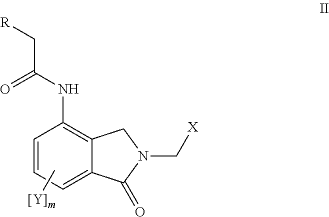

wherein:
R is aryl or cycloalkyl, optionally substituted with one or more R¹;

R¹ is halo, hydroxy, lower alkyl, lower haloalkyl, lower hydroxyalkyl, lower alkoxy, lower thioalkyl, lower alkyl sulfonyl, or lower haloalkoxy;

X is lower alkyl, optionally substituted with one or more X¹;

X¹ is halo, hydroxy, lower alkyl, lower haloalkyl, lower hydroxyalkyl, or lower alkoxy;

Y is halo;
m is 0, 1, or 2;
or a pharmaceutically acceptable salt thereof.

The application provides a compound selected from the group consisting of:
1-(1,3-Dioxo-2-pyridin-4-ylmethyl-2,3-dihydro-1H-isoindol-4-yl)-piperidine-3-carboxylic acid 2-methoxy-benzylamide;
(R)-1-(1,3-Dioxo-2-pyridin-4-ylmethyl-2,3-dihydro-1H-isoindol-4-yl)-piperidine-3-carboxylic acid 2-methoxy-benzylamide;
(S)-1-(1,3-Dioxo-2-pyridin-4-ylmethyl-2,3-dihydro-1H-isoindol-4-yl)-piperidine-3-carboxylic acid 2-methoxy-benzylamide;
(R)-1-(1,3-Dioxo-2-pyridin-4-ylmethyl-2,3-dihydro-1H-isoindol-4-yl)-piperidine-3-carboxylic acid (adamantan-1-ylmethyl)-amide;
(R)-1-(1,3-Dioxo-2-pyridin-4-ylmethyl-2,3-dihydro-1H-isoindol-4-yl)-piperidine-3-carboxylic acid (2-methoxy-phenyl)-amide;
(R)-1-[1,3-Dioxo-2-(tetrahydro-pyran-4-ylmethyl)-2,3-dihydro-1H-isoindol-4-yl]-piperidine-3-carboxylic acid 2-methoxy-benzylamide;
(R)-1-(1,3-Dioxo-2-thiazol-5-ylmethyl-2,3-dihydro-1H-isoindol-4-yl)-piperidine-3-carboxylic acid 2-methoxy-benzylamide;
(R)-1-(1,3-Dioxo-2-pyridin-4-ylmethyl-2,3-dihydro-1H-isoindol-4-yl)-piperidine-3-carboxylic acid 2-trifluoromethoxy-benzylamide;
(R)-1-(1,3-Dioxo-2-pyridin-4-ylmethyl-2,3-dihydro-1H-isoindol-4-yl)-piperidine-3-carboxylic acid (thiophen-2-ylmethyl)-amide;
(R)-1-(1,3-Dioxo-2-pyridin-4-ylmethyl-2,3-dihydro-1H-isoindol-4-yl)-piperidine-3-carboxylic acid 2-ethoxy-benzylamide;
(R)-1-(1,3-Dioxo-2-pyridin-4-ylmethyl-2,3-dihydro-1H-isoindol-4-yl)-piperidine-3-carboxylic acid 2,3-dimethoxy-benzylamide;
(R)-1-(1,3-Dioxo-2-pyridin-4-ylmethyl-2,3-dihydro-1H-isoindol-4-yl)-piperidine-3-carboxylic acid 2-hydroxy-benzylamide;
(R)-1-(1,3-Dioxo-2-pyridin-4-ylmethyl-2,3-dihydro-1H-isoindol-4-yl)-piperidine-3-carboxylic acid 2-methylsulfanyl-benzylamide;
(R)-1-(1,3-Dioxo-2-pyridin-4-ylmethyl-2,3-dihydro-1H-isoindol-4-yl)-piperidine-3-carboxylic acid 2-fluoro-benzylamide;
(R)-1-(1,3-Dioxo-2-pyridin-4-ylmethyl-2,3-dihydro-1H-isoindol-4-yl)-piperidine-3-carboxylic acid ([1,4]dioxan-2-ylmethyl)-amide;
(R)-1-(1,3-Dioxo-2-pyridin-4-ylmethyl-2,3-dihydro-1H-isoindol-4-yl)-piperidine-3-carboxylic acid cyclohexylmethyl-amide;
(R)-1-(1,3-Dioxo-2-pyridin-4-ylmethyl-2,3-dihydro-1H-isoindol-4-yl)-piperidine-3-carboxylic acid 2-methanesulfonyl-benzylamide;
N-[5-Chloro-2-((R)-2-hydroxy-1-methyl-ethyl)-1-oxo-2,3-dihydro-1H-isoindol-4-yl]-2-(4-fluoro-3-trifluoromethyl-phenyl)-acetamide;
2-Adamantan-1-yl-N-[5-chloro-2-((R)-2-hydroxy-1-methyl-ethyl)-1-oxo-2,3-dihydro-1H-isoindol-4-yl]-acetamide;
2-Adamantan-1-yl-N-[5,7-dichloro-2-((R)-2-hydroxy-1-methyl-ethyl)-1-oxo-2,3-dihydro-1H-isoindol-4-yl]-acetamide;
2-(4-Fluoro-3-trifluoromethyl-phenyl)-N-[2-(2-methoxy-1-methyl-ethyl)-1-oxo-2,3-dihydro-1H-isoindol-4-yl]-acetamide;
N-[5-Chloro-2-(2-methoxy-ethyl)-1-oxo-2,3-dihydro-1H-isoindol-4-yl]-2-(4-fluoro-3-trifluoromethyl-phenyl)-acetamide; and
N-[5-Chloro-2-((R)-2-methoxy-1-methyl-ethyl)-1-oxo-2,3-dihydro-1H-isoindol-4-yl]-2-(4-fluoro-3-trifluoromethyl-phenyl)-acetamide.

The application provides a pharmaceutical composition comprising:
(a) a pharmaceutically acceptable carrier; and
(b) a compound of Formula I or Formula II.

The application provides a method for treating arthritis, said method comprising administering to a subject in need thereof a therapeutically effective amount of a compound of Formula I or Formula II.

The application provides a method for treating a pain condition selected from inflammatory pain, surgical pain, visceral pain, dental pain, premenstrual pain, central pain, pain due to burns, migraine or cluster headaches, nerve injury, neuritis, neuralgias, poisoning, ischemic injury, interstitial cystitis, cancer pain, viral, parasitic or bacterial infection, post-traumatic injury, or pain associated with irritable bowel syndrome, said method comprising administering to a subject in need thereof a therapeutically effective amount of a compound of Formula I or Formula II.

The application provides a method for treating a respiratory disorder selected from chronic obstructive pulmonary disorder (COPD), asthma, and bronchospasm, said method comprising administering to a subject in need thereof a therapeutically effective amount of a compound of Formula I or Formula II.

The application provides a method for treating diabetes, said method comprising administering to a subject in need thereof a therapeutically effective amount of a compound of Formula I or Formula II.

The application provides the use of a compound of Formula I or Formula II in the preparation of a medicament for the treatment of autoimmune and inflammatory diseases associated with P2X7 modulation.

A compound, method, or use as described herein.

### Compounds

In general, the nomenclature used in this Application is based on AUTONOM™ v.4.0, a Beilstein Institute computerized system for the generation of IUPAC systematic nomenclature. Chemical structures shown herein were prepared using ISIS® version 2.2. Any open valency appearing on a carbon, oxygen sulfur or nitrogen atom in the structures herein indicates the presence of a hydrogen atom unless indicated otherwise. Where a nitrogen-containing heteroaryl ring is shown with an open valency on a nitrogen atom, and variables such as R^{a}, R^{b} or R^{c} are shown on the heteroaryl ring, such variables may be bound or joined to the open valency nitrogen. Where a chiral center exists in a structure but no specific stereochemistry is shown for the chiral center, both enantiomers associated with the chiral center are encompassed by the structure. Where a structure shown herein may exist in multiple tautomeric forms, all such tautomers are encompassed by the structure.

Examples of representative compounds encompassed by the present invention and within the scope of the invention are provided in the following Table. These examples and preparations which follow are provided to enable those skilled in the art to more clearly understand and to practice the present invention. They should not be considered as limiting the scope of the invention, but merely as being illustrative and representative thereof.

In general, the nomenclature used in this Application is based on AUTONOM™ v.4.0, a Beilstein Institute computerized system for the generation of IUPAC systematic nomenclature. If there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

TABLE I depicts examples of compounds according to generic Formula I or Formula II.

TABLE I

| | Nomenclature | Structure |
|---|---|---|
| I-1a | 1-(1,3-Dioxo-2- pyridin-4-ylmethyl- 2,3-dihydro-1H- isoindol-4-yl)- piperidine-3- carboxylic acid 2- methoxy- benzylamide | 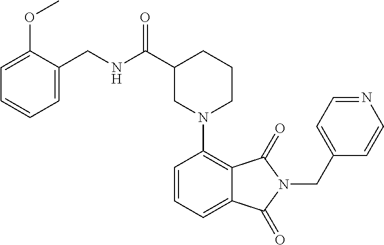 |
| I-1b | (R)-1-(1,3-Dioxo- 2-pyridin-4- ylmethyl-2,3- dihydro-1H- isoindol-4-yl)- piperidine-3- carboxylic acid 2- methoxy- benzylamide | 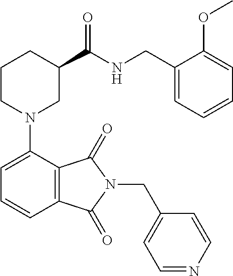 |
| I-2 | (S)-1-(1,3-Dioxo-2- pyridin-4-ylmethyl- 2,3-dihydro-1H- isoindol-4-yl)- piperidine-3- carboxylic acid 2- methoxy- benzylamide | 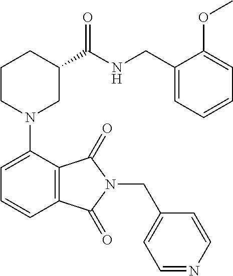 |
| I-3 | (R)-1-(1,3-Dioxo- 2-pyridin-4- ylmethyl-2,3- dihydro-1H- isoindol-4-yl)- piperidine-3- carboxylic acid (adamantan-1- ylmethyl)-amide | 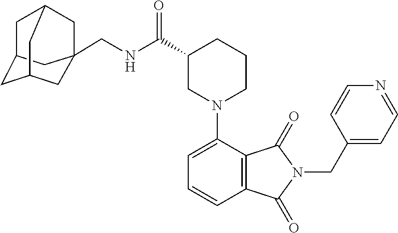 |
| I-4 | (R)-1-(1,3-Dioxo- 2-pyridin-4- ylmethyl-2,3- dihydro-1H- isoindol-4-yl)- piperidine-3- carboxylic acid (2- methoxy-phenyl)- amide | 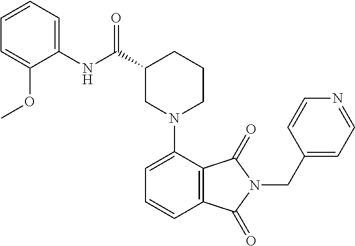 |
| I-5 | (R)-1-[1,3-Dioxo- 2-(tetrahydro- pyran-4-ylmethyl)- 2,3-dihydro-1H- isoindol-4-yl]- piperidine-3- carboxylic acid 2- methoxy- benzylamide | 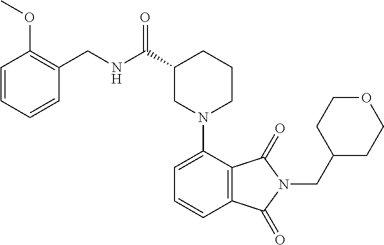 |
| I-6 | (R)-1-(1,3-Dioxo- 2-thiazol-5- ylmethyl-2,3- dihydro-1H- isoindol-4-yl)- piperidine-3- carboxylic acid 2- methoxy- benzylamide | 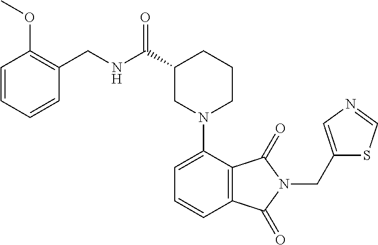 |
| I-7 | (R)-1-(1,3-Dioxo- 2-pyridin-4- ylmethyl-2,3- dihydro-1H- isoindol-4-yl)- piperidine-3- carboxylic acid 2- trifluoromethoxy- benzylamide | 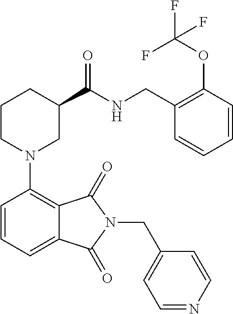 |
| I-8 | (R)-1-(1,3-Dioxo- 2-pyridin-4- ylmethyl-2,3- dihydro-1H- isoindol-4-yl)- piperidine-3- carboxylic acid (thiophen-2- ylmethyl)-amide | 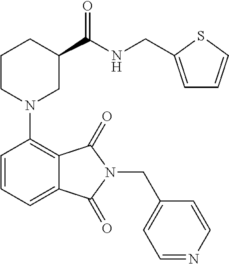 |
| I-9 | (R)-1-(1,3-Dioxo- 2-pyridin-4- ylmethyl-2,3- dihydro-1H- isoindol-4-yl)- piperidine-3- carboxylic acid 2- ethoxy- benzylamide | 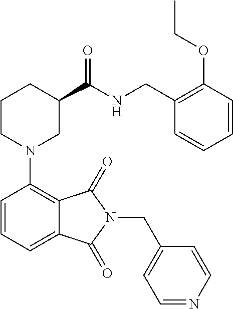 |
| I-10 | (R)-1-(1,3-Dioxo- 2-pyridin-4- ylmethyl-2,3- dihydro-1H- isoindol-4-yl)- piperidine-3- carboxylic acid 2,3- dimethoxy- benzylamide | 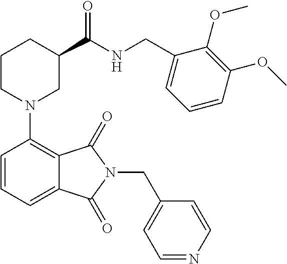 |
| I-11 | (R)-1-(1,3-Dioxo- 2-pyridin-4- ylmethyl-2,3- dihydro-1H- isoindol-4-yl)- piperidine-3- carboxylic acid 2- hydroxy- benzylamide | 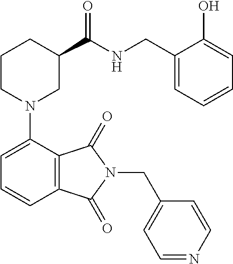 |
| I-12 | (R)-1-(1,3-Dioxo- 2-pyridin-4- ylmethyl-2,3- dihydro-1H- isoindol-4-yl)- piperidine-3- carboxylic acid 2- methylsulfanyl- benzylamide | 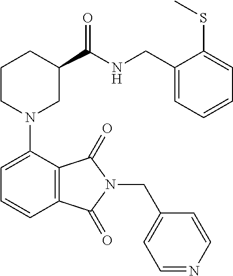 |
| I-13 | (R)-1-(1,3-Dioxo- 2-pyridin-4- ylmethyl-2,3- dihydro-1H- isoindol-4-yl)- piperidine-3- carboxylic acid 2- fluoro-benzylamide | 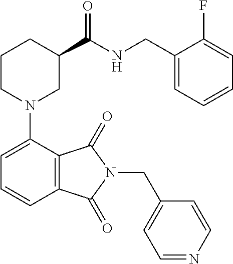 |
| I-14 | (R)-1-(1,3-Dioxo- 2-pyridin-4- ylmethyl-2,3- dihydro-1H- isoindol-4-yl)- piperidine-3- carboxylic acid ([1,4]dioxan-2- ylmethyl)-amide | 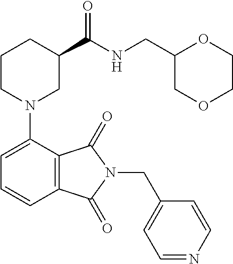 |
| I-15 | (R)-1-(1,3-Dioxo- 2-pyridin-4- ylmethyl-2,3- dihydro-1H- isoindol-4-yl)- piperidine-3- carboxylic acid cyclohexylmethyl- amide | 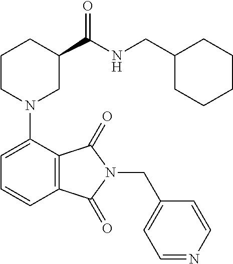 |
| I-16 | (R)-1-(1,3-Dioxo- 2-pyridin-4- ylmethyl-2,3- dihydro-1H- isoindol-4-yl)- piperidine-3- carboxylic acid 2- methanesulfonyl- benzylamide | 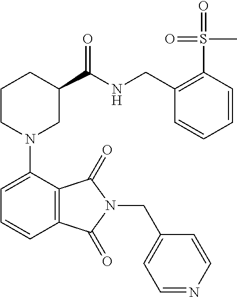 |
| I-17 | N-[5-Chloro-2- ((R)-2-hydroxy-1- methyl-ethyl)-1- oxo-2,3-dihydro- 1H-isoindol-4-yl]- 2-(4-fluoro-3- trifluoromethyl- phenyl)-acetamide | 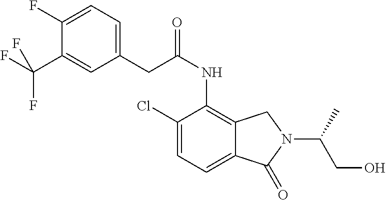 |
| I-18a | 2-Adamantan-1-yl- N-[5-chloro-2-((R)- 2-hydroxy-1- methyl-ethyl)-1- oxo-2,3-dihydro- 1H-isoindol-4-yl]- acetamide | 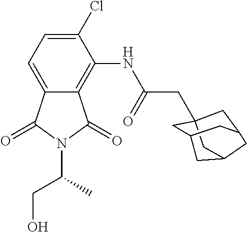 |
| I-18b | 2-Adamantan-1-yl- N-[5,7-dichloro-2- ((R)-2-hydroxy-1- methyl-ethyl)-1- oxo-2,3-dihydro- 1H-isoindol-4-yl]- acetamide | 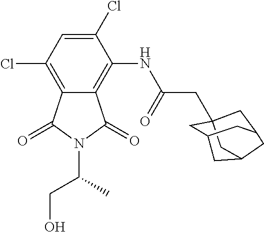 |
| I-19 | 2-(4-Fluoro-3- trifluoromethyl- phenyl)-N-[2-(2- methoxy-1-methyl- ethyl)-1-oxo-2,3- dihydro-1H- isoindol-4-yl]- acetamide | 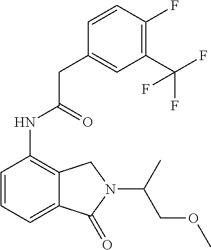 |
| I-20 | N-[5-Chloro-2-(2- methoxy-ethyl)-1- oxo-2,3-dihydro- 1H-isoindol-4-yl]- 2-(4-fluoro-3- trifluoromethyl- phenyl)-acetamide | 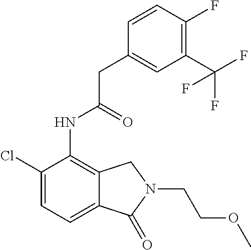 |
| I-21 | N-[5-Chloro-2- ((R)-2-methoxy-1- methyl-ethyl)-1- oxo-2,3-dihydro- 1H-isoindol-4-yl]- 2-(4-fluoro-3- trifluoromethyl- phenyl)-acetamide | 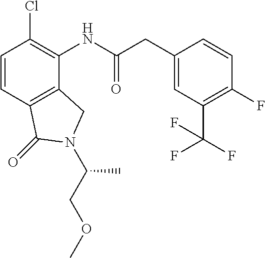 |

### Synthesis

### General Schemes

### Examples 1-4, 7-16

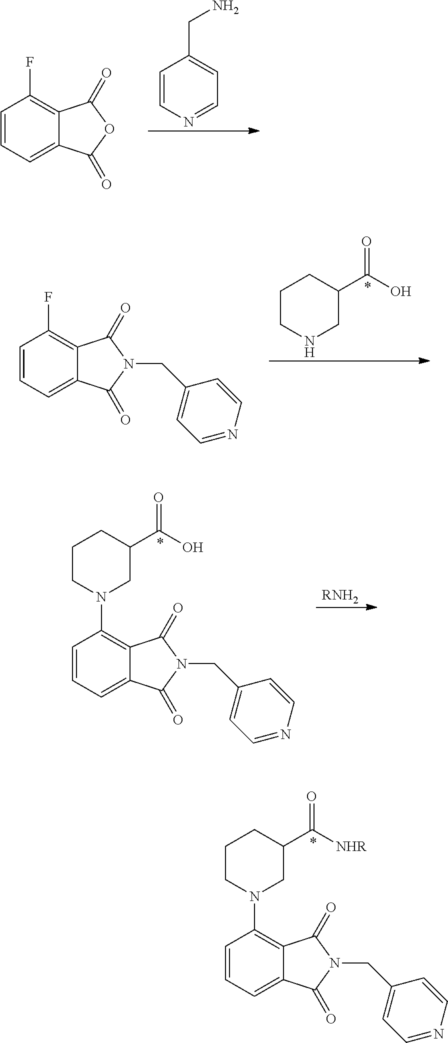

In the above General Scheme, R can be R′ or CH₂R′, R′ can be aryl, cycloalkyl, heterocycloalkyl, heteroaryl, optionally substituted with one or more R¹, R¹ can be halo, hydroxy, lower alkyl, lower haloalkyl, lower hydroxyalkyl, lower alkoxy, lower thioalkyl, lower alkyl sulfonyl, or lower haloalkoxy, X can be lower alkyl, aryl, cycloalkyl, heterocycloalkyl, or heteroaryl, optionally substituted with one or more X¹, and X¹ can be halo, hydroxy, lower alkyl, lower haloalkyl, lower hydroxyalkyl, or lower alkoxy.

### Examples 5-6

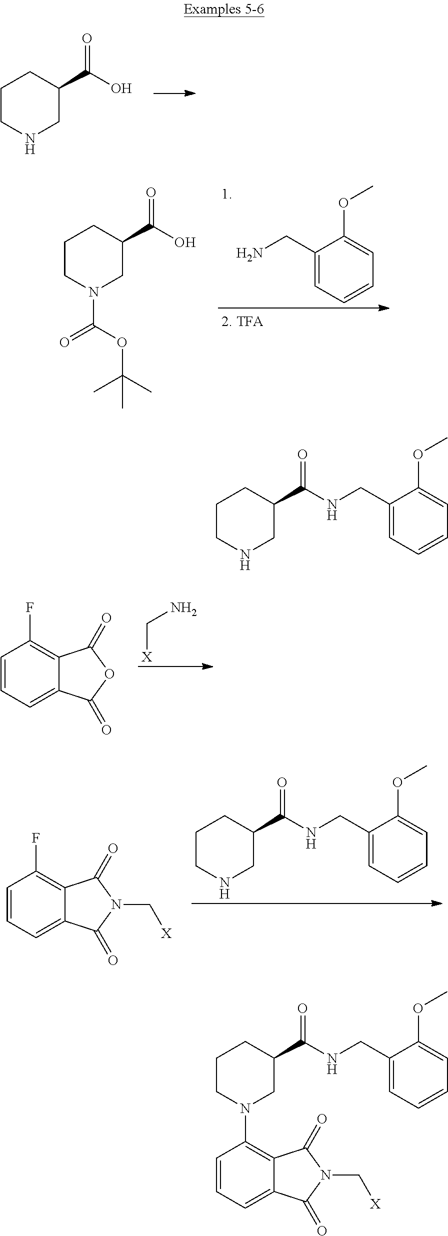

In the above General Scheme, X can be lower alkyl, aryl, cycloalkyl, heterocycloalkyl, or heteroaryl, optionally substituted with one or more X¹, and X¹ can be halo, hydroxy, lower alkyl, lower haloalkyl, lower hydroxyalkyl, or lower alkoxy.

### Examples 17-21

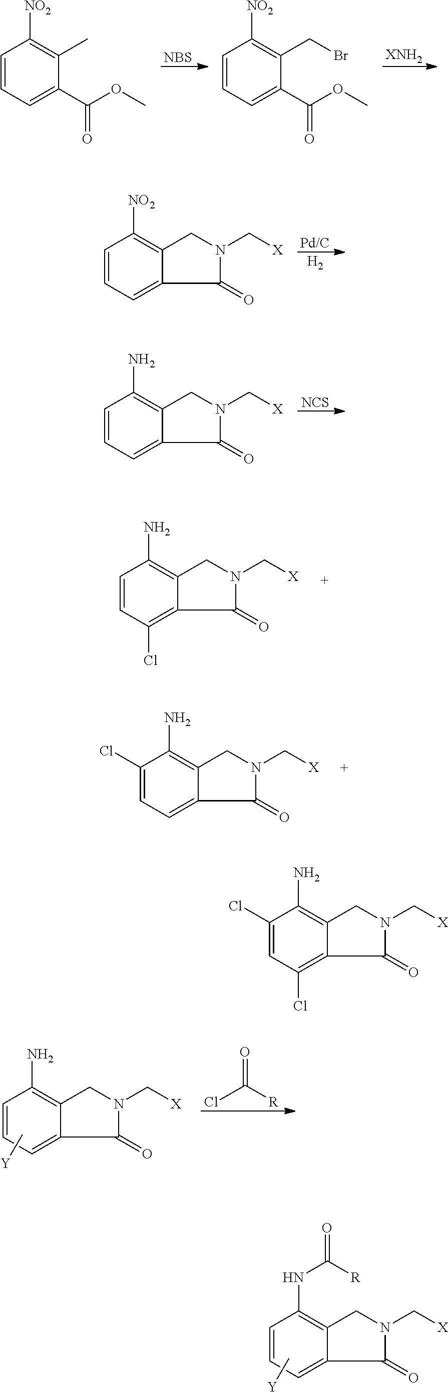

In the above General Schemes, R can be R′ or CH₂R′, R′ can be aryl or cycloalkyl, optionally substituted with one or more R¹, R¹ can be halo, hydroxy, lower alkyl, lower haloalkyl, lower hydroxyalkyl, lower alkoxy, lower thioalkyl, lower alkyl sulfonyl, or lower haloalkoxy, X can be lower alkyl, optionally substituted with one or more X¹, X¹ can be halo, hydroxy, lower alkyl, lower haloalkyl, lower hydroxyalkyl, or lower alkoxy, and Y is halo.

### Pharmaceutical Compositions and Administration

### Dosage and Administration

The compounds of the present invention may be formulated in a wide variety of oral administration dosage forms and carriers. Oral administration can be in the form of tablets, coated tablets, dragées, hard and soft gelatin capsules, solutions, emulsions, syrups, or suspensions. Compounds of the present invention are efficacious when administered by other routes of administration including continuous (intravenous drip) topical parenteral, intramuscular, intravenous, subcutaneous, transdermal (which may include a penetration enhancement agent), buccal, nasal, inhalation and suppository administration, among other routes of administration. The preferred manner of administration is generally oral using a convenient daily dosing regimen which can be adjusted according to the degree of affliction and the patient's response to the active ingredient.

A compound or compounds of the present invention, as well as their pharmaceutically useable salts, together with one or more conventional excipients, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. A typical preparation will contain from about 5% to about 95% active compound or compounds (w/w). The term “preparation” or “dosage form” is intended to include both solid and liquid formulations of the active compound and one skilled in the art will appreciate that an active ingredient can exist in different preparations depending on the target organ or tissue and on the desired dose and pharmacokinetic parameters.

The term “excipient” as used herein refers to a compound that is useful in preparing a pharmaceutical composition, generally safe, non-toxic and neither biologically nor otherwise undesirable, and includes excipients that are acceptable for veterinary use as well as human pharmaceutical use. The compounds of this invention can be administered alone but will generally be administered in admixture with one or more suitable pharmaceutical excipients, diluents or carriers selected with regard to the intended route of administration and standard pharmaceutical practice.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

A “pharmaceutically acceptable salt” form of an active ingredient may also initially confer a desirable pharmacokinetic property on the active ingredient which were absent in the non-salt form, and may even positively affect the pharmacodynamics of the active ingredient with respect to its therapeutic activity in the body. The phrase “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, colorants, flavors, stabilizers, buffers, artificial and natural sweeteners, dispersants, thickeners, solubilizing agents, and the like.

Liquid formulations also are suitable for oral administration include liquid formulation including emulsions, syrups, elixirs, aqueous solutions, aqueous suspensions. These include solid form preparations which are intended to be converted to liquid form preparations shortly before use. Emulsions may be prepared in solutions, for example, in aqueous propylene glycol solutions or may contain emulsifying agents such as lecithin, sorbitan monooleate, or acacia. Aqueous solutions can be prepared by dissolving the active component in water and adding suitable colorants, flavors, stabilizing, and thickening agents. Aqueous suspensions can be prepared by dispersing the finely divided active component in water with viscous material, such as natural or synthetic gums, resins, methylcellulose, sodium carboxymethylcellulose, and other well known suspending agents.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilization from solution for constitution before use with a suitable vehicle, e.g., sterile, pyrogen-free water.

The compounds of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the present invention may be formulated for vaginal administration. Pessaries, tampons, creams, gels, pastes, foams or sprays containing in addition to the active ingredient such carriers as are known in the art to be appropriate.

The compounds of the present invention may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the present invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to an skin-adhesive solid support. The compound of interest can also be combined with a penetration enhancer, e.g., Azone (1-dodecylaza-cycloheptan-2-one). Sustained release delivery systems are inserted subcutaneously into to the subdermal layer by surgery or injection. The subdermal implants encapsulate the compound in a lipid soluble membrane, e.g., silicone rubber, or a biodegradable polymer, e.g., polylactic acid.

Suitable formulations along with pharmaceutical carriers, diluents and excipients are described in Remington: The Science and Practice of Pharmacy 1995, edited by E. W. Martin, Mack Publishing Company, 19th edition, Easton, Pa. A skilled formulation scientist may modify the formulations within the teachings of the specification to provide numerous formulations for a particular route of administration without rendering the compositions of the present invention unstable or compromising their therapeutic activity.

The modification of the present compounds to render them more soluble in water or other vehicle, for example, may be easily accomplished by minor modifications (salt formulation, esterification, etc.), which are well within the ordinary skill in the art. It is also well within the ordinary skill of the art to modify the route of administration and dosage regimen of a particular compound in order to manage the pharmacokinetics of the present compounds for maximum beneficial effect in patients.

The term “therapeutically effective amount” as used herein means an amount required to reduce symptoms of the disease in an individual. The dose will be adjusted to the individual requirements in each particular case. That dosage can vary within wide limits depending upon numerous factors such as the severity of the disease to be treated, the age and general health condition of the patient, other medicaments with which the patient is being treated, the route and form of administration and the preferences and experience of the medical practitioner involved. For oral administration, a daily dosage of between about 0.01 and about 1000 mg/kg body weight per day should be appropriate in monotherapy and/or in combination therapy. A preferred daily dosage is between about 0.1 and about 500 mg/kg body weight, more preferred 0.1 and about 100 mg/kg body weight and most preferred 1.0 and about 10 mg/kg body weight per day. Thus, for administration to a 70 kg person, the dosage range would be about 7 mg to 0.7 g per day. The daily dosage can be administered as a single dosage or in divided dosages, typically between 1 and 5 dosages per day. Generally, treatment is initiated with smaller dosages which are less than the optimum dose of the compound. Thereafter, the dosage is increased by small increments until the optimum effect for the individual patient is reached. One of ordinary skill in treating diseases described herein will be able, without undue experimentation and in reliance on personal knowledge, experience and the disclosures of this application, to ascertain a therapeutically effective amount of the compounds of the present invention for a given disease and patient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Pharmaceutical preparations for delivery by various routes are formulated as shown in the following Tables. “Active ingredient” or “Active compound” as used in the Tables means one or more of the Compounds of Formula I or II.

Composition for Oral Administration

| Ingredient | % wt./wt. |
|---|---|
| Active ingredient | 20.0% |
| Lactose | 79.5% |
| Magnesium stearate | 0.5% |

The ingredients are mixed and dispensed into capsules containing about 100 mg each; one capsule would approximate a total daily dosage.

Composition for Oral Administration

| Ingredient | % wt./wt. |
|---|---|
| Active ingredient | 20.0% |
| Magnesium stearate | 0.5% |
| Crosscarmellose sodium | 2.0% |
| Lactose | 76.5% |
| PVP (polyvinylpyrrolidine) | 1.0% |

The ingredients are combined and granulated using a solvent such as methanol. The formulation is then dried and formed into tablets (containing about 20 mg of active compound) with an appropriate tablet machine.

Composition for Oral Administration

| Ingredient | Amount | |
|---|---|---|
| Active compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum K (Vanderbilt Co.) | 1.0 | g |
| Flavoring | 0.035 | ml |
| Colorings | 0.5 | mg |
| Distilled water | q.s. to 100 | ml |

The ingredients are mixed to form a suspension for oral administration.

Parenteral Formulation

| Ingredient | % wt./wt. |
|---|---|
| Active ingredient | 0.25 g |
| Sodium Chloride | q.s. to make isotonic |
| Water for injection | 100 ml |

The active ingredient is dissolved in a portion of the water for injection. A sufficient quantity of sodium chloride is then added with stirring to make the solution isotonic. The solution is made up to weight with the remainder of the water for injection, filtered through a 0.2 micron membrane filter and packaged under sterile conditions.

Suppository Formulation

| Ingredient | % wt./wt. |
|---|---|
| Active ingredient | 1.0% |
| Polyethylene glycol 1000 | 74.5% |
| Polyethylene glycol 4000 | 24.5% |

The ingredients are melted together and mixed on a steam bath, and poured into molds containing 2.5 g total weight.

Topical Formulation

| Ingredients | Grams |
|---|---|
| Active compound | 0.2-2 |
| Span 60 | 2 |
| Tween 60 | 2 |
| Mineral oil | 5 |
| Petrolatum | 10 |
| Methyl paraben | 0.15 |
| Propyl paraben | 0.05 |
| BHA (butylated hydroxy anisole) | 0.01 |
| Water | q.s. 100 |

All of the ingredients, except water, are combined and heated to about 60° C. with stirring. A sufficient quantity of water at about 60° C. is then added with vigorous stirring to emulsify the ingredients, and water then added q.s. about 100 g.

### Nasal Spray Formulations

Several aqueous suspensions containing from about 0.025-0.5 percent active compound are prepared as nasal spray formulations. The formulations optionally contain inactive ingredients such as, for example, microcrystalline cellulose, sodium carboxymethylcellulose, dextrose, and the like. Hydrochloric acid may be added to adjust pH. The nasal spray formulations may be delivered via a nasal spray metered pump typically delivering about 50-100 microliters of formulation per actuation. A typical dosing schedule is 2-4 sprays every 4-12 hours.

### Indications and Methods of Treatment

The compounds of the invention are usable for the treatment of a wide range of inflammatory diseases and conditions such as arthritis, including but not limited to, rheumatoid arthritis, spondyloarthropathies, gouty arthritis, osteoarthritis, systemic lupus erythematosus and juvenile arthritis, and other arthritic conditions. The subject compounds would be useful for the treatment of pulmonary disorders or lung inflammation, including adult respiratory distress syndrome, pulmonary sarcoidosis, asthma, silicosis, and chronic pulmonary inflammatory disease.

The compounds of the invention are also expected to find utility as analgesics in the treatment of diseases and conditions associated with pain from a wide variety of causes, including, but not limited to, inflammatory pain such as pain associated with arthritis (including rheumatoid arthritis and osteoarthritis), surgical pain, visceral pain, dental pain, premenstrual pain, central pain, pain due to burns, migraine or cluster headaches, nerve injury, neuritis, neuralgias, poisoning, ischemic injury, interstitial cystitis, cancer pain, viral, parasitic or bacterial infection, post-traumatic injuries (including fractures and sports injuries), and pain associated with functional bowel disorders such as irritable bowel syndrome.

Further, compounds of the invention are useful for treating respiratory disorders, including chronic obstructive pulmonary disorder (COPD), asthma, bronchospasm, and the like.

Additionally, compounds of the invention are useful for treating gastrointestinal disorders, including Irritable Bowel Syndrome (IBS), Inflammatory Bowel Disease (IBD), biliary colic and other biliary disorders, renal colic, diarrhea-dominant IBS, pain associated with GI distension, and the like.

The compounds of the invention are also useful for the treatment of muscular sclerosis and diabetes.

The application provides a method for treating arthritis, said method comprising administering to a subject in need thereof a therapeutically effective amount of a compound of Formula I or Formula II.

The application provides a method for treating a pain condition selected from inflammatory pain, surgical pain, visceral pain, dental pain, premenstrual pain, central pain, pain due to burns, migraine or cluster headaches, nerve injury, neuritis, neuralgias, poisoning, ischemic injury, interstitial cystitis, cancer pain, viral, parasitic or bacterial infection, post-traumatic injury, or pain associated with irritable bowel syndrome, said method comprising administering to a subject in need thereof a therapeutically effective amount of a compound of Formula I or Formula II.

The application provides a method for treating a respiratory disorder selected from chronic obstructive pulmonary disorder (COPD), asthma, and bronchospasm, said method comprising administering to a subject in need thereof a therapeutically effective amount of a compound of Formula I or Formula II.

The application provides a method for treating diabetes, said method comprising administering to a subject in need thereof a therapeutically effective amount of a compound of Formula I or Formula II.

### EXAMPLES

The following preparations and examples are given to enable those skilled in the art to more clearly understand and to practice the present invention. They should not be considered as limiting the scope of the invention, but merely as being illustrative and representative thereof.

### ABBREVIATIONS

The following abbreviations may be used in the Preparations and Examples.
AIBN 2,2′azobis(2-methylpropionitrile)
BETBDMS 2-bromoethoxy tertbutyldimethylsilane
BOP (benzotriazol-1-yloxy)tris(dimethylamino)phosphonium hexafluorophosphate
DBU 1,8-diazabicyclo[5.4.0]undec-7-ene
DCM dichloromethane/methylene chloride
DIPEA diisopropyl ethylamine (Hunig's base)
DME 1,2-dimethoxyethane (glyme)
DMF N,N-dimethylformamide
DMFDMA N,N-dimethylformamide dimethyl acetal
DMSO dimethyl sulfoxide
DMAP 4-dimethylaminopyridine
dppf 1,1′-Bis(diphenylphosphino)ferrocene
EDCI 1-ethyl-3-(3′-dimethylaminopropyl)carbodiimide
EDC.HCl 1-[3-(dimethylamino)propyl]-3-ethylcarbodiimide hydrochloride
EtOAc ethyl acetate
EtOH ethanol
gc gas chromatography
HATU O-(7-Azabenzotriazol-1-yl)-N,N,N′,N′-tetramethyluronium hexafluorophosphate
HMPA hexamethylphosphoramide
HOAc acetic acid
HOBt N-Hydroxybenzotriazole
hplc high performance liquid chromatography
IPA isopropanol
IPBAPE isopropenylboronic acid pinacol ester
mCPBA m-chloroperbenzoic acid
MeCN acetonitrile
NBS N-bromosuccinimide
NCS N-chlorosuccinimide
NMM N-methyl morpholine
NMP N-methylpyrrolidinone
Pd₂(dba)₃ Tris(dibenzylideneacetone)dipalladium(0)
TBAF tetra-n-butyl ammonium fluoride
tBDMSICl tert-butyl-dimethylsilyl chloride
TEA triethylamine
TFA trifluoroacetic acid
THF tetrahydrofuran
LDA lithium diisopropylamine
TBDMS tert-butyl dimethylsilyl chloride
TLC thin layer chromatography
Xantphos 4,5-bis(diphenylphosphino)-9,9-dimethyl-xanthene

### General Conditions

Unless otherwise stated, all temperatures including melting points (i.e., MP) are in degrees Celsius (° C.). It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

Compounds of the present invention can be prepared beginning with commercially available starting materials, unless where the source of the material is specifically stated, or utilizing general synthetic techniques and procedures known to those skilled in the art. Chemicals may be purchased from companies such as for example Aldrich, Argonaut Technologies, VWR, Lancaster, Princeton, Alfa, Oakwood, TCI, Fluorochem, Apollo, Matrix, Maybridge or Meinoah. Chromatography supplies and equipment may be purchased from such companies as for example AnaLogix, Inc, Burlington, Wis.; Biotage AB, Charlottesville, Va.; Analytical Sales and Services, Inc., Pompton Plains, N.J.; Teledyne Isco, Lincoln, Nebr.; VWR International, Bridgeport, N.J.; Varian Inc., Palo Alto, Calif., and Multigram II Mettler Toledo Instrument Newark, Del. Biotage, ISCO and Analogix columns are pre-packed silica gel columns used in standard chromatography. The intermediate and final compounds were named using the AutoNom2000 feature as part of the MDL ISIS Draw application. NMR data was collected using a Bruker NMR.

### PREPARATIVE EXAMPLES

### Example 1

### (R)-1-(1,3-Dioxo-2-pyridin-4-ylmethyl-2,3-dihydro-1H-isoindol-4-yl)-piperidine-3-carboxylic acid 2-methoxy-benzylamide

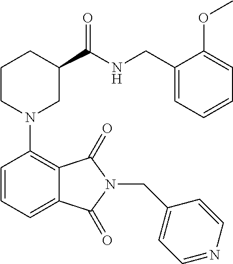

A mixture of 4-fluoro-isobenzofuran-1,3-dione (1.66 g, 10 mmol) and pyridin-4-yl-methylamine (1.08 g, 10 mmol) in 10 mL acetic acid was stirred at reflux for 2 h. The reaction mixture was cooled to room temperature, and added to a solution of 10 g of Na₂CO₃ in H₂O. The resulting mixture was extracted with ethyl acetate. The combined extracts were dried over MgSO₄, filtered and concentrated in vacuo. The crude product was passed through a pad of silica and eluted with ethyl acetate to afford 4-fluoro-2-pyridin-4-ylmethyl-isoindole-1,3-dione (2.41 g, 94%) of as a white crystalline solid: LC/MS-ESI observed [M+H]⁺ 257.

A mixture of 4-fluoro-2-pyridin-4-ylmethyl-isoindole-1,3-dione (0.51 g, 2 mmol), (R)-piperidine-3-carboxylic acid (0.516 g, 4 mmol) and cesium carbonate (1.3 g, 4 mmol) in 8 mL of DMSO was stirred under N₂ at 100° C. for 1.5 h. The mixture was cooled to room temperature, diluted with H₂O (20 mL) and the resulting mixture neutralized with 0.45 g of acetic acid. The resulting mixture was extracted with CHCl₃. The CHCl₃ extracts were dried over MgSO₄, filtered and concentrated in vacuo. The resulting oil was triturated with ether to afford a solid, which was filtered and washed several times with ether followed by hexanes to afford (R)-1-(1,3-dioxo-2-pyridin-4-ylmethyl-2,3-dihydro-1H-isoindol-4-yl)-piperidine-3-carboxylic acid (0.61 g, 84%) as a yellow crystalline solid.

A mixture of (R)-1-(1,3-dioxo-2-pyridin-4-ylmethyl-2,3-dihydro-1H-isoindol-4-yl)-piperidine-3-carboxylic acid (0.2 g, 0.55 mmol), EDC.HCl (0.116 g, 0.605 mmol) and DMAP (8 mg) in 8 mL of CH₂Cl₂ was stirred at room temperature for 5 minutes and then cooled to 0° C. 2-Methoxybenzylamine (0.091 g, 0.66 mmol) was added. The resulting mixture was allowed to warm slowly to room temperature and stirred overnight. The mixture was diluted with 8 mL of ether, washed with 5% aqueous NaOH, dried over Na₂SO₄ and filtered through a pad of silica. The silica was eluted with 5% methanol in CHCl₃. The yellow band was collected and concentrated to give a bright yellow solid. Recrystallization from CHCl₃-ether gave (R)-1-(1,3-dioxo-2-pyridin-4-ylmethyl-2,3-dihydro-1H-isoindol-4-yl)-piperidine-3-carboxylic acid 2-methoxy-benzylamide (0.162 g, 61%) as a bright yellow crystalline solid: LC/MS-ESI observed [M+H]⁺ 485.

### Example 2

### (S)-1-(1,3-Dioxo-2-pyridin-4-ylmethyl-2,3-dihydro-1H-isoindol-4-yl)-piperidine-3-carboxylic acid 2-methoxy-benzylamide

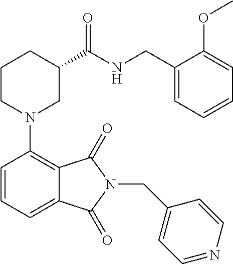

(S)-1-(1,3-Dioxo-2-pyridin-4-ylmethyl-2,3-dihydro-1H-isoindol-4-yl)-piperidine-3-carboxylic acid was prepared as described in example 1, except substituting (S)-piperidine-3-carboxylic acid for (R)-piperidine-3-carboxylic acid to afford (S)-1-(1,3-dioxo-2-pyridin-4-ylmethyl-2,3-dihydro-1H-isoindol-4-yl)-piperidine-3-carboxylic acid (0.66 g, 90%) as a yellow solid.

To a stirred mixture of (S)-1-(1,3-dioxo-2-pyridin-4-ylmethyl-2,3-dihydro-1H-isoindol-4-yl)-piperidine-3-carboxylic acid (0.2 g, 0.55 mmol) and TEA (0.056 g, 0.55 mmol) in THF (7 mL) were added 2-methoxybenzylamine (0.077 g, 0.56 mmol) and BOP (0.25 g, 0.56 mmol). The reaction mixture was stirred at room temperature overnight. The resultant reaction mixture was diluted with CH₂Cl₂, washed with 1.5 M aqueous. Na₂CO₃, dried over MgSO₄, filtered and concentrated in vacuo to afford a yellow solid. The solid was dissolved in a small amount of CH₂Cl₂ and passed through a thick pad of silica (5% methanol in CHCl₃ eluant) to afford a yellow oil. Crystallization from ether afforded (S)-1-(1,3-dioxo-2-pyridin-4-ylmethyl-2,3-dihydro-1H-isoindol-4-yl)-piperidine-3-carboxylic acid 2-methoxy-benzylamide (0.26 g, 98%) as a yellow crystalline solid: LC/MS-ESI observed [M+H]⁺ 485.

### Example 3

### (R)-1-(1,3-Dioxo-2-pyridin-4-ylmethyl-2,3-dihydro-1H-isoindol-4-yl)-piperidine-3-carboxylic acid (adamantan-1-ylmethyl)-amide

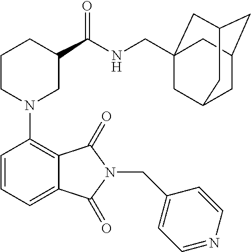

(R)-1-(1,3-dioxo-2-pyridin-4-ylmethyl-2,3-dihydro-1H-isoindol-4-yl)-piperidine-3-carboxylic acid was prepared as described in example 1 (0.66 g, 90%).

To (R)-1-(1,3-dioxo-2-pyridin-4-ylmethyl-2,3-dihydro-1H-isoindol-4-yl)-piperidine-3-carboxylic acid (0.34 g, 0.93 mmol) and TEA (0.094 g, 0.93 mmol) in THF (7 mL) were added adamantan-1-ylmethylamine (0.16 g, 0.96 mmol) and BOP (0.425 g, 0.96 mmol). The reaction mixture was stirred at room temperature overnight, then concentrated to reduced volume and passed through a thick pad of silica (5% methanol in CHCl₃ eluant) to afford (R)-1-(1,3-dioxo-2-pyridin-4-ylmethyl-2,3-dihydro-1H-isoindol-4-yl)-piperidine-3-carboxylic acid (adamantan-1-ylmethyl)-amide (475 mg, 100%) as a yellow foam: LC/MS-ESI observed [M+H]⁺ 513.

### Example 4

### (R)-1-(1,3-Dioxo-2-pyridin-4-ylmethyl-2,3-dihydro-1H-isoindol-4-yl)-piperidine-3-carboxylic acid (2-methoxyphenyl)-amide

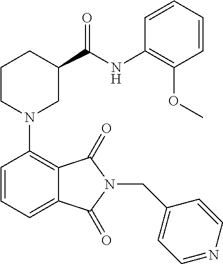

(R)-1-(1,3-dioxo-2-pyridin-4-ylmethyl-2,3-dihydro-1H-isoindol-4-yl)-piperidine-3-carboxylic acid was prepared on a 3 mmol scale as described in example 1 (0.98 g, 89%).

To (R)-1-(1,3-dioxo-2-pyridin-4-ylmethyl-2,3-dihydro-1H-isoindol-4-yl)-piperidine-3-carboxylic acid (0.183 g, 0.5 mmol), o-anisidine (0.062 g, 0.5 mmol) and TEA (0.051 g, 0.5 mmol) in THF (7 mL) was added BOP (0.22 g, 0.5 mmol). The reaction mixture was stirred at room temperature for 1.5 h and then at 55° C. for 3 h, then concentrated in vacuo. Purification on an AnaLogix Intelliflash system (80 g column, silica SF40, 0-2% methanol/chloroform) followed by crystallization from ethyl acetate-ether afforded (R)-1-(1,3-Dioxo-2-pyridin-4-ylmethyl-2,3-dihydro-1H-isoindol-4-yl)-piperidine-3-carboxylic acid (2-methoxyphenyl)-amide (170 mg, 72%) as a yellow foam: LC/MS-ESI observed [M+H]⁺ 471.

### Example 5

### (R)-1-[1,3-Dioxo-2-(tetrahydro-pyran-4-ylmethyl)-2,3-dihydro-1H-isoindol-4-yl]-piperidine-3-carboxylic acid 2-methoxy-benzylamide

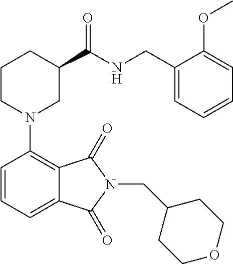

To a mixture of (R)-piperidine-3-carboxylic acid (0.65 g, 5 mmol) and TEA (0.51 g, 5 mmol) in CHCl₃ (25 mL) was added di-t-butyldicarbonate (1.2 g, 5.5 mmol). The resulting mixture was stirred at 60° C. for 1 h. The mixture was cooled to room temperature, washed with 5% HCl, dried over MgSO₄, filtered and concentrated in vacuo. The resulting solid was suspended in hexanes and filtered. The solid was washed with hexanes and dried to afford 1.07 g of a white crystalline solid. The solid was dissolved in dry THF (20 mL). To that was added with stirring TEA (0.473 g, 4.7 mmol), 2-methoxybenzylamine (0.66 g, 4.8 mmol) and BOP (2.12 g, 4.8 mmol). The resulting reaction mixture was stirred at room temperature overnight and then concentrated in vacuo. Purification on an AnaLogix Intelliflash system (80 g column, silica, 15-50% ethyl acetate/hexanes) afforded 1.44 g of a colorless oil, which was dissolved in CH₂Cl₂ (5 mL) and treated with TFA (3 mL). The resulting solution was stirred at room temperature for 30 min., diluted with 50 mL of a 1:1 mixture of ether: CH₂Cl₂ and made basic with 7 mL of 25% aqueous NaOH. The layers were separated and the aqueous layer was extracted with a 1:1 mixture of ether: CH₂Cl₂. The combined organic extracts were washed with saturated NaCl, dried over MgSO₄, filtered and concentrated in vacuo. The resulting colorless resin was triturated with ether to give (R)-piperidine-3-carboxylic acid 2-methoxy-benzylamide (0.92 g, 74%) as a white crystalline solid: LC/MS-ESI observed [M+H]⁺ 249.

A mixture of 4-fluoro-isobenzofuran-1,3-dione (0.5 g, 3 mmol) and (tetrahydropyran-4-yl)methylamine (0.35 g, 3 mmole) in 4 mL acetic acid was stirred at reflux for 4 h. The reaction mixture was concentrated in vacuo. The resulting tan solid was recrystallized from ethyl acetate to afford 4-fluoro-2-(tetrahydro-pyran-4-ylmethyl)-isoindole-1,3-dione (0.59 g, 75%) of as a beige crystalline solid.

4-Fluoro-2-(tetrahydro-pyran-4-ylmethyl)-isoindole-1,3-dione (0.13 g, 0.5 mmol), (R)-piperidine-3-carboxylic acid 2-methoxy-benzylamide (0.15 g, 0.6 mmol) and cesium carbonate (0.195 g, 0.6 mmol) were combined in 2 mL of DMSO. The reaction mixture was stirred at 100° C. for 3 h. The mixture was cooled to room temperature, diluted with CHCl₃, washed with H₂O, dried over MgSO4, filtered and concentrated in vacuo to give a yellow oil. The yellow oil was crystallized from benzene-hexanes to afford (R)-1-[1,3-dioxo-2-(tetrahydro-pyran-4-ylmethyl)-2,3-dihydro-1H-isoindol-4-yl]-piperidine-3-carboxylic acid 2-methoxy-benzylamide (0.177 g, 72%) of as a yellow crystalline solid: LC/MS-ESI observed [M+H]⁺ 492.

### Example 6

### (R)-1-(1,3-Dioxo-2-thiazol-5-ylmethyl-2,3-dihydro-1H-isoindol-4-yl)-piperidine-3-carboxylic acid 2-methoxy-benzylamide

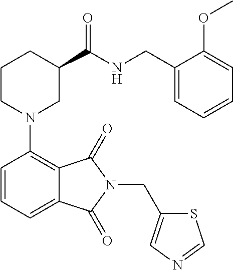

A mixture of 4-fluoro-isobenzofuran-1,3-dione (0.62 g, 3.73 mmol) and thiazol-5-ylmethyl-carbamic acid tert-butyl ester (0.89 g of 90% pure, 3.73 mmole) in 5 mL acetic acid was stirred at reflux for 2.5 h. The reaction mixture was concentrated in vacuo. The residue was taken up in ethyl acetate, washed with 5% aqueous NaOH, dried over Mg₂SO₄, filtered and concentrated in vacuo to afford a tan oil. Trituration with ether/hexanes afforded 4-fluoro-2-thiazol-5-ylmethyl-isoindole-1,3-dione (0.52 g, 53%) as a tan crystalline solid: LC/MS-ESI observed [M+H]⁺ 263.

4-Fluoro-2-thiazol-5-ylmethyl-isoindole-1,3-dione (0.21 g, 0.8 mmol), (R)-piperidine-3-carboxylic acid 2-methoxy-benzylamide (prepared in example 5, 0.25 g, 1 mmol) and cesium carbonate (0.33 g, 1 mmol) were combined in 3 mL of DMSO. The reaction mixture was stirred at 85° C. overnight. The mixture was diluted with H₂O and extracted several times with CH₂Cl₂:ether (1:1). The combined extracts were washed with H₂O: saturated NaCl (3:1), dried over anhydrous K₂CO₃, filtered and concentrated to a yellow solid. The solid was dissolved in CHCl₃ and passed through a short column of silica (2% methanol in CHCl₃ eluant). The resulting solid was crystallized from CHCl₃-ether to afford (R)-1-(1,3-dioxo-2-thiazol-5-ylmethyl-2,3-dihydro-1H-isoindol-4-yl)-piperidine-3-carboxylic acid 2-methoxy-benzylamide (0.09 g, 23%); as a yellow solid: LC/MS-ESI observed [M+H]⁺ 491.

### Example 7

### (R)-1-(1,3-Dioxo-2-pyridin-4-ylmethyl-2,3-dihydro-1H-isoindol-4-yl)-piperidine-3-carboxylic acid 2-trifluoromethoxybenzylamide

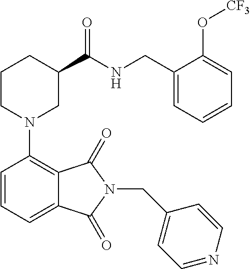

A mixture of 4-fluoro-isobenzofuran-1,3-dione (5 g, 30.1 mmol) and pyridin-4-yl-methylamine (3.06 mL, 30.3 mmole) in 30 mL acetic acid was stirred at reflux for 2 h. The reaction mixture was concentrated in vacuo. The residue was taken up in 100 mL saturated Na₂CO₃ and 200 mL CHCl3 and the layers were separated. The organic phase was dried over Na₂SO₄ and concentrated in vacuo. The residue was dissolved in 300 mL of ethyl acetate and 18 g silica was added. The mixture was filtered and the filtrate concentrated in vacuo. The resulting white solid was triturated with hexanes to afford 4-fluoro-2-pyridin-4-ylmethyl-isoindole-1,3-dione (6.87 g, 89%) as a white solid.

A mixture of 4-fluoro-2-pyridin-4-ylmethyl-isoindole-1,3-dione (2 g, 7.8 mmol), (R)-piperidine-3-carboxylic acid (1.2 g, 9.2 mmol) and cesium carbonate (4 g, 11.7 mmol) in 15 mL of DMSO was stirred at 105° C. for 2 h. 100 mL of H₂O was added, followed by 4.0 mL of acetic acid and the mixture was placed in a sonicator (Branson 2510) for 10 minutes. The yellow solid was collected, washed with 5 mL of H₂O and dried in vacuo to afford (R)-1-(1,3-Dioxo-2-pyridin-4-ylmethyl-2,3-dihydro-1H-isoindol-4-yl)-piperidine-3-carboxylic acid (2.6 g, 91%).

To a stirred mixture of (R)-1-(1,3-Dioxo-2-pyridin-4-ylmethyl-2,3-dihydro-1H-isoindol-4-yl)-piperidine-3-carboxylic acid (0.3 g, 0.82 mmol) and TEA (0.35 mL, 2.5 mmol) in THF (7 mL) were added 2-trifluoromethoxybenzylamine (0.114 g, 1.0 mmol) and BOP (0.400 g, 0.9 mmol). The reaction mixture was stirred at room temperature for 2 h, then diluted with 5 mL NaHCO₃ and 15 mL of H₂O. The product was filtered off as yellow needles, washed with 50% aqueous THF and dried to afford (R)-1-(1,3-dioxo-2-pyridin-4-ylmethyl-2,3-dihydro-1H-isoindol-4-yl)-piperidine-3-carboxylic acid 2-trifluoromethoxybenzylamide (0.401 g, 83%) as fine yellow needles: m.p. 197-198° C., LC/MS-ESI observed [M+H]⁺ 539.

### Example 8

### (R)-1-(1,3-Dioxo-2-pyridin-4-ylmethyl-2,3-dihydro-1H-isoindol-4-yl)-piperidine-3-carboxylic acid (thiophen-2-ylmethyl)amide

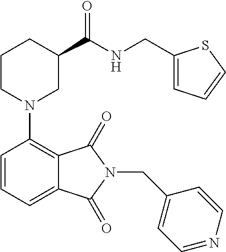

To a stirred mixture of (R)-1-(1,3-Dioxo-2-pyridin-4-ylmethyl-2,3-dihydro-1H-isoindol-4-yl)-piperidine-3-carboxylic acid (prepared in example 7, 0.3 g, 0.82 mmol) and TEA (0.35 mL, 2.5 mmol) in THF (7 mL) were added 2-aminomethylthiophene (0.114 g, 1.0 mmol) and BOP (0.400 g, 0.9 mmol). The reaction mixture was stirred at room temperature for 2 h, then diluted with 10 mL of saturated NaCl, 5 mL saturated NaHCO₃ and 15 mL of H₂O and extracted with ethyl acetate. The combined extracts were dried over MgSO₄, filtered and concentrated in vacuo. The product was recrystallized from ethyl acetate:hexane to afford (R)-1-(1,3-dioxo-2-pyridin-4-ylmethyl-2,3-dihydro-1H-isoindol-4-yl)-piperidine-3-carboxylic acid (thiophen-2-ylmethyl)amide (300 mg, 79%) as yellow crystals: m.p. 193-194° C., LC/MS-ESI observed [M+H]⁺ 461.

### Example 9

### (R)-1-(1,3-Dioxo-2-pyridin-4-ylmethyl-2,3-dihydro-1H-isoindol-4-yl)-piperidine-3-carboxylic acid 2-ethoxybenzylamide

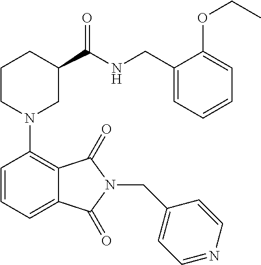

Preparation by a similar procedure to Example 8, except substituting 2-ethoxybenzylamine for 2-aminomethylthiophene, afforded (R)-1-(1,3-dioxo-2-pyridin-4-ylmethyl-2,3-dihydro-1H-isoindol-4-yl)piperidine-3-carboxylic acid 2-ethoxybenzyl-amide (337 mg, 82%) as yellow crystals; m.p. 194-195° C., LC/MS-ESI observed [M+H]⁺ 499.

### Example 10

### (R)-1-(1,3-Dioxo-2-pyridin-4-ylmethyl-2,3-dihydro-1H-isoindol-4-yl)-piperidine-3-carboxylic acid 2,3-dimethoxybenzylamide

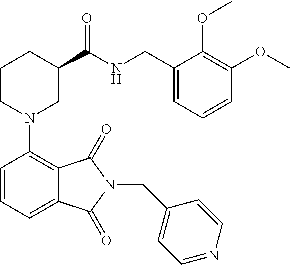

Preparation by a similar procedure to Example 8, except substituting 2,3-dimethoxy-benzylamine for 2-aminomethylthiophene, afforded (R)-1-(1,3-dioxo-2-pyridin-4-ylmethyl-2,3-dihydro-1H-isoindol-4-yl)piperidine-3-carboxylic acid 2,3-dimethoxybenzylamide (227 mg, 54%) as yellow crystals; m.p. 199-200° C., LC/MS-ESI observed [M+H]⁺ 515.

### Example 11

### (R)-1-(1,3-Dioxo-2-pyridin-4-ylmethyl-2,3-dihydro-1H-isoindol-4-yl)-piperidine-3-carboxylic acid 2-hydroxybenzylamide

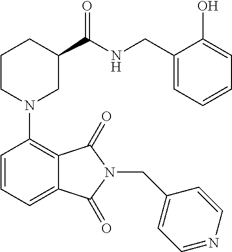

A mixture of 4-fluoro-2-pyridin-4-ylmethyl-isoindole-1,3-dione (2 g, 7.8 mmol), (R)-piperidine-3-carboxylic acid (1.2 g, 9.2 mmol) and cesium carbonate (4 g, 11.7 mmol) in 15 mL of DMSO was stirred at 105° C. for 2 h. 100 mL of H₂O was added. The mixture was slowly poured into 4.0 mL of acetic acid with stirring. The yellow precipitate was collected, washed with 5 mL of H₂O and dried in vacuo to afford (R)-1-(1,3-Dioxo-2-pyridin-4-ylmethyl-2,3-dihydro-1H-isoindol-4-yl)-piperidine-3-carboxylic acid (2.61 g, 91%).

Preparation by a similar procedure to Example 8, except substituting 2-hydroxybenzyl-amine for 2-aminomethylthiophene, afforded (R)-1-(1,3-dioxo-2-pyridin-4-ylmethyl-2,3-dihydro-1H-isoindol-4-yl)piperidine-3-carboxylic acid 2-hydroxybenzylamide (329 mg, 85%) as a yellow solid after recrystallization from methanol: water; m.p. 181-182° C., LC/MS-ESI observed [M+H]⁺ 471.

### Sample 12

(R)-1-(1,3-Dioxo-2-pyridin-4-ylmethyl-2,3-dihydro-1H-isoindol-4-yl)-piperidine-3-carboxylic acid 2-(methylthio)benzylamide

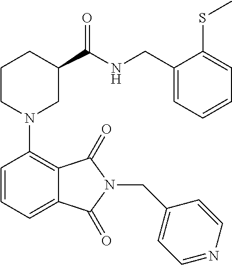

Preparation by a similar procedure to Example 8, except substituting 2-(methylthio)-benzylamine for 2-aminomethylthiophene, afforded (R)-1-(1,3-dioxo-2-pyridin-4-ylmethyl-2,3-dihydro-1H-isoindol-4-yl)piperidine-3-carboxylic acid 2-(methylthio)-benzylamide (401 mg, 98%) as a yellow solid after trituration with methanol; m.p. 216-217° C., LC/MS-ESI observed [M+H]⁺ 501.

### Example 13

### (R)-1-(1,3-Dioxo-2-pyridin-4-ylmethyl-2,3-dihydro-1H-isoindol-4-yl)-piperidine-3-carboxylic acid 2-fluorobenzylamide

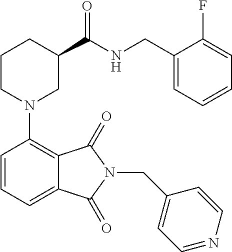

Preparation by a similar procedure to Example 8, except substituting 2-fluorobenzyl-amine for 2-aminomethylthiophene, afforded (R)-1-(1,3-dioxo-2-pyridin-4-ylmethyl-2,3-dihydro-1H-isoindol-4-yl)piperidine-3-carboxylic acid 2-fluorobenzylamide (335 mg, 86%) as a yellow solid after trituration with methanol; m.p. 196-197° C., LC/MS-ESI observed [M+H]⁺ 473.

### Example 14

### (R)-1-(1,3-Dioxo-2-pyridin-4-ylmethyl-2,3-dihydro-1H-isoindol-4-yl)-piperidine-3-carboxylic acid ([1,4]dioxan-2-ylmethyl)-amide

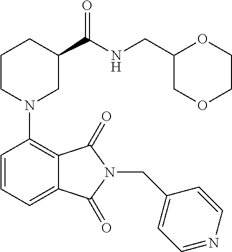

Preparation by a similar procedure to Example 8, except substituting [1,4]Dioxan-2-yl-methylamine for 2-aminomethylthiophene, afforded (R)-1-(1,3-dioxo-2-pyridin-4-ylmethyl-2,3-dihydro-1H-isoindol-4-yl)piperidine-3-carboxylic acid ([1,4]dioxan-2-ylmethyl)amide (205 mg, 54%) as a yellow solid after recrystallization from methanol; m.p. 186-187° C., LC/MS-ESI observed [M+H]⁺ 465.

### Example 15

### (R)-1-(1,3-Dioxo-2-pyridin-4-ylmethyl-2,3-dihydro-1H-isoindol-4-yl)-piperidine-3-carboxylic acid cyclohexylmethylamide

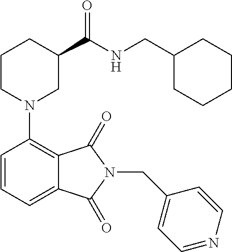

Preparation by a similar procedure to Example 8, except substituting aminomethylcyclo-hexane for 2-aminomethylthiophene, afforded (R)-1-(1,3-dioxo-2-pyridin-4-ylmethyl-2,3-dihydro-1H-isoindol-4-yl)piperidine-3-carboxylic acid cyclohexylmethylamide (243 mg, 54%) as a yellow solid after trituration with methanol; m.p. 172-173° C., LC/MS-ESI observed [M+H]⁺ 461.

### Example 16

### (R)-1-(1,3-Dioxo-2-pyridin-4-ylmethyl-2,3-dihydro-1H-isoindol-4-yl)-piperidine-3-carboxylic acid 2-methanesulfonylbenzylamide

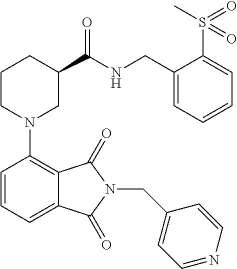

Prepared by a similar procedure to that of (R)-1-(1,3-dioxo-2-pyridin-4-ylmethyl-2,3-dihydro-1H-isoindol-4-yl)-piperidine-3-carboxylic acid (thiophen-2-ylmethyl)-amide except substituting 2-methanesulfonylbenzylamine for 2-aminomethylthiophene to afford 270 mg (R)-1-(1,3-dioxo-2-pyridin-4-ylmethyl-2,3-dihydro-1H-isoindol-4-yl)piperidine-3-carboxylic acid 2-methanesulfonylbenzylamide as a yellow solid after recrystallization from methanol; followed by chromatography (silica, 80% ethyl acetate: 20% (0.5% acetic acid in hexanes) eluant); m.p. 101-102° C., LC/MS-ESI observed [M+H]⁺ 533.

### Example 17

### N-[5-Chloro-2-((R)-2-hydroxy-1-methyl-ethyl)-1-oxo-2,3-dihydro-1H-isoindol-4-yl]-2-(4-fluoro-3-trifluoromethylphenyl)acetamide

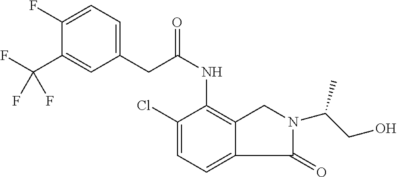

A mixture of methyl 2-methyl-3-nitrobenzoate (5 g, 25.6 mmole), AIBN (100 mg) and NBS (4.62 g, 26 mmol) in 50 mL of CH₂Cl₂ was heated under reflux for 18 h. Reaction was not complete. Additional NBS (2 g, 11.2 mmol) was added and the mixture was heated under reflux for an additional 18 h. Additional NBS (3.5 g, 19.7 mmol) was added and the reaction was stirred at room temperature for 2 days. The mixture was filtered and the filtrate concentrated in vacuo. Purification on an AnaLogix Intelliflash system (24 g column, SF15 silica, 5-10% ethyl acetate/hexanes) afforded pure (150 mg) and mixed (6.4 g) fractions. Re-purification on an AnaLogix Intelliflash system (200 g column, SF65 silica, 5-10% ethyl acetate/hexanes) afforded 2-bromomethyl-3-nitro-benzoic acid

methyl ester (2.43 g, 34%); ¹H NMR (300 MHz, CDCl₃) δ ppm 4.00 (s, 3H), 5.16 (s, 2H), 7.54 (t, J=8 Hz, 1H), 7.97 (dd, J=1.4, 7.9, 1H), 8.11 (dd, J=1.4, 7.9, 1H).

To a solution of 2-bromomethyl-3-nitro-benzoic acid methyl ester (500 mg, 1.82 mmol) in 10 mL CHCl₃ was added a solution of D-alaminol (300 mg, 4 mmol) in 1 mL of CHCl₃. The resulting solution was stirred at room temperature for 30 min and at 70° C. for 18 h. The reaction mixture was allowed to cool to room temperature, diluted with 20 mL H₂O and extracted with CHCl₃. The CHCl₃ layer was dried over Na₂SO₄ and concentrated in vacuo. Trituration with ether provided 2-((R)-2-hydroxy-1-methyl-ethyl)-4-nitro-2,3-dihydroisoindol-1-one (317 mg, 74% yield).

2-((R)-2-hydroxy-1-methyl-ethyl)-4-nitro-2,3-dihydroisoindol-1-one (240 mg, 1.02 mmol) and 40 mg 10% Pd/C in 15 mL of methanol was hydrogenated at 50 psi at room temperature for 1 hr. The catalyst was filtered off (Whatman GF/B paper), washed with methanol and concentrated in vacuo to afford 4-amino-2-((R)-2-hydroxy-1-methylethyl)-2,3-dihydroisoindol-1-one (200 mg, 100%).

4-amino-2-((R)-2-hydroxy-1-methylethyl)-2,3-dihydroisoindol-1-one (200 mg, 1 mmol) and N-chlorosuccinimide (140 mg, 1.05 mmol) were combined in 5 mL of acetonitrile and stirred at 25° C. for 1 hr and 75° C. for 1 hr. Purification on an AnaLogix Intelliflash system (12 g, SF15 silica, 1000:900:120:15 hexanes:ethyl acetate: methanol: NH₄OH) afforded 4-amino-5-chloro-2-((R)-2-hydroxy-1-methylethyl)-2,3-dihydro-isoindol-1-one (82 mg); ¹H NMR (300 MHz, DMSO-d6) δ ppm 1.16 (d, J=7.0 Hz, 3H), 3.55 (m), 4.25 (m, s, 3H), 6.87 (d, J=7.9, 1H), 7.34 (d, J=7.9, 1H). The lower spot from the column was isolated to afford 4-amino-7-chloro-2-((R)-2-hydroxy-1-methyl-ethyl)-2,3-dihydro-isoindol-1-one (84 mg); ¹H NMR (300 MHz, DMSO) δ ppm 1.16 (d, J=7 Hz, 3H), 3.55 (m), 4.18 (s, 2H), 4.28 (m, 1H), 6.74 (d, J=8.6, 1H), 7.11 (d, J=8.6, 1H).

To a solution of 4-fluoro-3-trifluoromethylphenyl acetic acid (3.0 g, 13.5 mmol) in 35 mL CH₂Cl₂ was added 2 drops of DMF and oxalyl chloride (2.0 mL, 23 mmol). The solution was stirred at room temperature for 1 h and then concentrated under reduced pressure to afford 4-fluoro-3-trifluoromethyl-phenyl)acetyl chloride (3.2 g) as a yellow oil.

4-Amino-5-chloro-2-((R)-2-hydroxy-1-methylethyl)-2,3-dihydro-isoindol-1-one (62 mg, 0.26 mmol), NaHCO₃ (84 mg, 1 mmol), dodecyltrimethyl ammonium bromide (2 mg) and 4-fluoro-3-trifluoromethylphenylacetyl chloride (75 mg, 0.31 mmol) were combined in 5 mL THF and stirred at reflux for 3 h. The solvent was removed under reduced pressure and the crude mixture was dissolved in 10 mL of methanol and 0.5 mL of H₂O. To this solution was added K₂CO₃ (200 mg, 1.45 mmol). The reaction mixture was stirred at room temperature for 18 h, diluted with 5 mL H₂O and extracted with 20 mL ethyl acetate. The organic extracts were dried over MgSO₄, filtered, and concentrated in vacuo. Purification on an AnaLogix Intelliflash system (12 g SF15 silica, 5:85:0.5:0.5 hexanes:ethyl acetate: methanol: NH₄OH) afforded N-[5-chloro-2-((R)-2-hydroxy-1-methyl-ethyl)-1-oxo-2,3-dihydro-1H-isoindol-4-yl]-2-(4-fluoro-3-trifluoromethylphenyl)-acetamide (40 mg, 35%) as a white foam; LC/MS-ESI observed [M+H]⁺ 445.

### Example 18

### 2-Adamantan-1-yl-N-[5-chloro-2-((R)-2-hydroxy-1-methyl-ethyl)-1-oxo-2,3-dihydro-1H-isoindol-4-yl]-acetamide and 2-Adamantan-1-yl-N-[5,7-dichloro-2-((R)-2-hydroxy-1-methyl-ethyl)-1-oxo-2,3-dihydro-1H-isoindol-4-yl]-acetamide

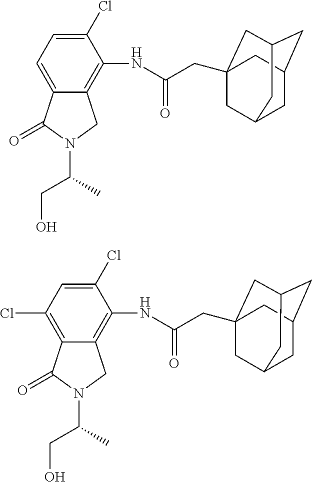

A mixture of methyl 2-methyl-3-nitrobenzoate (5 g, 25.6 mmol), NBS (5 g, 28.1 mmol) and benzoylperoxide (100 mg) in 50 mL of CCl₄ was heated under reflux for 18 h. The mixture was filtered and the filtrate concentrated under reduced pressure. The residue was recrystallized from ether to provide 2-bromomethyl-3-nitro-benzoic acid methyl ester (4.18 g, 60%) as light yellow crystals.

A solution of 2-bromomethyl-3-nitro-benzoic acid methyl ester (5.05 g, 18.1 mmol) in 40 mL toluene was stirred at 50° C. D-Alaminol (3.0 g, 40 mmol) was added and the reaction mixture was stirred at 50° C. for 6 h; then room temperature for 18 h. The solvent was removed under reduced pressure. The residue was added to 20 mL H₂O and 20 mL ether and the resultant mixture was stirred at room temperature for 0.5 h. The resultant white solid was collected and washed with H₂O, ether, and then hexanes and dried under vacuum to afford 2-((R)-2-hydroxy-1-methyl-ethyl)-4-nitro-2,3-dihydroisoindol-1-one (3.69 g, 86%) as a white solid.

To a solution of adamantan-1-yl-acetic acid (1.0 g, 5.1 mmol) in 20 mL CH₂Cl₂ was added 2 drops of DMF and oxalyl chloride (0.61 mL, 7 mmol). The solution was stirred at room temperature for 1 h and then concentrated under reduced pressure to afford adamantan-1-yl-acetyl chloride (1.1 g) as an oil.

A mixture of 2-((R)-2-hydroxy-1-methyl-ethyl)-4-nitro-2,3-dihydroisoindol-1-one (3.55 g, 15 mmol) and 10% Pd/C (500 mg) in 70 mL methanol was shaken under 50 psi hydrogen atmosphere for 1 h. The mixture was filtered (Whatman GF/B paper) and concentrated under reduced pressure. The resulting foam was stirred in 75 mL acetonitrile and heated to 75° C. N-chlorosuccinimide (2.1 g, 15.75 mmole) was added in portions over 20 minutes. The reaction mixture was stirred at 75° C. for 1 h. The solution was concentrated under reduced pressure and dissolved in 10 mL 10% methanol in CH₂Cl₂. Purification on an AnaLogix Intelliflash system (200 g SF65 silica, 1000:900:120:15 hexanes:ethyl acetate:methanol:NH₄OH) afforded the front peak, containing 4-amino-5-chloro-2-((R)-2-hydroxy-1-methylethyl)-2,3-dihydro-isoindol-1-one (1.28 g, 35%), and the back peak, containing 4-amino-7-chloro-2-((R)-2-hydroxy-1-methyl-ethyl)-2,3-dihydro-isoindol-1-one (1.61 g, 45%).

A portion of the front peak, containing 4-amino-5-chloro-2-((R)-2-hydroxy-1-methylethyl)-2,3-dihydro-isoindol-1-one (100 mg, 0.42 mmol), adamantan-1-yl-acetyl chloride (213 mg, 1.0 mmole), NaHCO₃ (250 mg, 3 mmol) and 10 mg dodecyl trimethyl ammonium bromide in 10 mL of THF was heated at reflux for 2.5 h. The mixture was concentrated under reduced pressure. The residue was stirred in a mixture of 7 mL methanol and 0.5 mL H₂O. K₂CO₃ (400 mg, 2.9 mmol) was added and the mixture was stirred at room temperature for 16 h. 4M aqueous NaOH was added and the reaction was stirred an additional 4 h at room temperature. The crude reaction mixture was concentrated under reduced pressure to remove the methanol and the residue was partitioned between 20 mL H₂O and extracted with ethyl acetate (2×25 mL). The organic extracts were dried over MgSO₄ and concentrated in vacuo. Purification on an AnaLogix Intelliflash system (24 g SF15 silica, 5:4.5:0.5:15 hexanes:ethyl acetate:methanol:acetic acid) afforded a minor front peak, containing 2-adamantan-1-yl-N-[5,7-dichloro-2-((R)-2-hydroxy-1-methyl-ethyl)-1-oxo-2,3-dihydro-1H-isoindol-4-yl]-acetamide (28 mg) as a foam: LC/MS-ESI observed [M+H]⁺ 451. The major product was isolated as the later peak to afford 2-adamantan-1-yl-N-[5-chloro-2-((R)-2-hydroxy-1-methyl-ethyl)-1-oxo-2,3-dihydro-1H-isoindol-4-yl]-acetamide (102 mg, 58%) as a foam: LC/MS-ESI observed [M+H]⁺ 417.

### Example 19

### 4-Fluoro-N-[2-(2-methoxy-1-methyl-ethyl)-1-oxo-2,3-dihydro-1H-isoindol-4-yl]-3-trifluoromethyl-benzamide

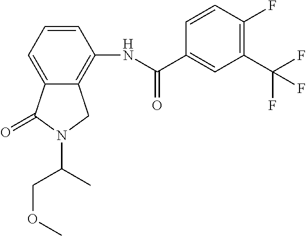

A mixture of methyl 2-methyl-3-nitrobenzoate (6.2 g, 32 mmol), NBS (6 g, 33.7 mmol) and benzoylperoxide (100 mg) in 50 mL of CCl₄ was heated under reflux for 4 h and 80° C. for 16 h. Reaction was 50% complete. Another 3 g (16.9 mmol) NBS and 100 mg benzoyl peroxide were added. The reaction was heated at reflux for an additional 3 h. The mixture was concentrated under reduced pressure. Ethyl acetate (10 mL) was added and mixture was filtered. The filtrate was concentrated under reduced pressure and the resulting residue was recrystallized from ether/hexanes to afford 2-bromomethyl-3-nitro-benzoic acid methyl ester (6.21 g). Purification of the mother liquor on an AnaLogix Intelliflash system (150 g SF40 silica, 5-10% ethyl acetate/hexanes) afforded a additional product (2.5 g). Overall yield was 99%.

A mixture of 2-bromomethyl-3-nitro-benzoicacid methyl ester (750 mg, 2.74 mmol) and 2-amino-1-methoxypropane (490 mg, 5.5 mmol) in 15 mL toluene was heated at reflux for 1.5 h. The reaction mixture was diluted with 10 mL H₂O and extracted with 20 mL of ethyl acetate. The organic phase was dried over MgSO₄ and concentrated under reduced pressure. Recrystallization of the resultant residue with ether/hexanes afforded 2-(2-methoxy-1-methyl-ethyl)-4-nitro-2,3-dihydro-isoindol-1-one (544 mg, 79%) as a white solid: LC/MS-ESI observed [M+H]⁺ 251.

A mixture of 2-(2-methoxy-1-methylethyl)-4-nitro-2,3-dihydro-isoindol-1-one (510 mg, 2.04 mmol) and 150 mg 10% Pd/C in 25 mL of methanol was shaken under 50 psi of hydrogen at room temperature for 45 minutes. The catalyst was removed by filtration through a Whatman GF/B filter and celite. The filtrate was concentrated under reduced pressure. Recrystallization from ethyl acetate/hexanes afforded 4-amino-2-(2-methoxy-1-methyl-ethyl)-2,3-dihydro-isoindol-1-one (460 mg, 100%).

To a stirred mixture of 2-(2-methoxy-1-methylethyl)-4-nitro-2,3-dihydro-isoindol-1-one (450 mg, 2.04 mmol) in 15 mL THF and 6 mL H₂O was added NaHCO₃ (500 mg, 6 mmol) and a solution of 4-fluoro-3-trifluoromethyl-phenyl)acetyl chloride (prepared in example 17, 600 mg, 2.5 mmol) in 2 mL THF. The reaction mixture was stirred at room temperature for 1.5 h. The reaction was diluted with 10 mL H₂O and 10 mL saturated NaCl and extracted with 25 mL ethyl acetate. The organic phase was dried over MgSO₄ and concentrated under reduced pressure. Recrystallization of the resulting solid from ethyl acetate/hexanes afforded 4-fluoro-N-[2-(2-methoxy-1-methyl-ethyl)-1-oxo-2,3-dihydro-1H-isoindol-4-yl]-3-trifluoromethyl-benzamide (673 mg, 78%): m.p. 151-152° C., LC/MS-ESI observed [M+H]⁺ 425.

### Example 20

### N-[5-Chloro-2-(2-methoxy-ethyl)-1-oxo-2,3-dihydro-1H-isoindol-4-yl]-4-fluoro-3-trifluoromethyl-benzamide

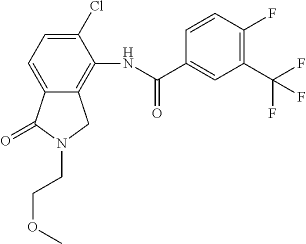

A solution of 2-bromomethyl-3-nitro-benzoic acid methyl ester (prepared as in example 20, 700 mg, 2.55 mmole) and 2-methoxyethylamine (250 mg, 3.33 mmol) were combined in 15 mL of acetonitrile and 5 mL H₂O and heated at reflux for 1 h. The reaction mixture was concentrated under reduced pressure to 6 mL. The residue was partitioned between 20 mL ethyl acetate and 10 mL saturated NaCl. The organic phase was dried over MgSO₄ and concentrated under reduced pressure. Recrystallization from ether/hexanes afforded 2-(2-methoxyethyl)-4-nitro-2,3-dihydro-isoindol-1-one (380 mg, 63%). The reaction was repeated using different conditions. A solution of 2-bromomethyl-3-nitro-benzoic acid methyl ester (500 mg, 1.82 mmol) and 2-methoxyethylamine (300 mg, 4 mmol) in 10 mL toluene was heated at reflux for 1 h. The crude reaction mixture was diluted with 5 mL saturated NaHCO₃ and 5 mL H₂O. The mixture was extracted with 25 mL ethyl acetate. The aqueous phase was saturated with solid NaCl and extracted with 10 mL ethyl acetate. The combined organic phase was dried over MgSO₄ and concentrated under reduced pressure. Recrystallization from ether/hexanes afforded additional 2-(2-methoxyethyl)-4-nitro-2,3-dihydro-isoindol-1-one (380 mg, 88% yield).

A mixture of 2-(2-methoxyethyl)-4-nitro-2,3-dihydro-isoindol-1-one (720 mg, 3.05 mmol) and 190 mg 10% Pd/C in 20 mL of methanol was shaken under 50 psi of hydrogen at room temperature for 30 minutes. The catalyst was removed by filtration. The filtrate was concentrated under reduced pressure. Crystallization from ether, followed by washing with hexanes afforded 4-amino-2-(2-methoxy-1-ethyl)-2,3-dihydro-isoindol-1-one (533 mg, 85%).

A solution of 4-amino-2-(2-methoxy-1-ethyl)-2,3-dihydro-isoindol-1-one (500 mg, 2.42 mmol) and NCS (360 mg, 2.7 mmol) in 10 mL acetonitrile was heated at reflux for 1 h. LCMS showed a 54:32:13 ratio of monochloro-product:monochloro-product:dichloro-product. The reaction mixture was concentrated under reduced pressure. Purification on an AnaLogix Intelliflash system (40 g SF25 silica, 5:4.5:0.5:0.5 hexanes:ethyl acetate:methanol:NH₄OH) afforded a front peak. This solvent was evaporated and the residue partitioned between 10 mL ethyl acetate and 3 mL H₂O to remove residual succinimide. The organic phase was dried over MgSO4 and concentrated in vacuo. to afforded a 75:25 mixture of 4-amino-5-chloro-2-(2-methoxyethyl)-2,3-dihydro-isoindol-1-one and 4-amino-5,7-dichloro-2-(2-methoxyethyl)-2,3-dihydro-isoindol-1-one (260 mg). The late-running peak from the column was concentrated to afford 4-amino-7-chloro-2-(2-methoxy-ethyl)-2,3-dihydro-isoindol-1-one (270 mg).

The 75:25 mixture of 4-amino-5-chloro-2-(2-methoxy-ethyl)-2,3-dihydro-isoindol-1-one and 4-amino-5,7-dichloro-2-(2-methoxy-ethyl)-2,3-dihydro-isoindol-1-one (260 mg, 1.08 mmol), NaHCO₃ (250 mg, 2.98 mmol) and 4-fluoro-3-trifluoromethyl-phenyl)acetyl chloride (prepared in example 17, 290 mg, 1.2 mmol) and 30 mg dodecyl trimethyl ammonium bromide in 10 mL of THF was heated at reflux for 1 h. The mixture was diluted with 6 mL of H₂O, 5 mL saturated NaCl and extracted with 20 mL ethyl acetate. The organic phase was dried over MgSO₄ and concentrated under reduced pressure. Purification on an AnaLogix Intelliflash system (40 g SF25 silica, 7:2.5:0.5:0.5 hexanes:ethyl acetate: methanol: acetic acid) afforded N-[5-chloro-2-(2-methoxy-ethyl)-1-oxo-2,3-dihydro-1H-isoindol-4-yl]-4-fluoro-3-trifluoromethylbenzamide (146 mg) as a white foam: LC/MS-ESI observed [M+H]⁺ 445.

### Example 21

### N-[5-Chloro-2-((R)-2-methoxy-1-methyl-ethyl)-1-oxo-2,3-dihydro-1H-isoindol-4-yl]-4-fluoro-3-trifluoromethyl-benzamide

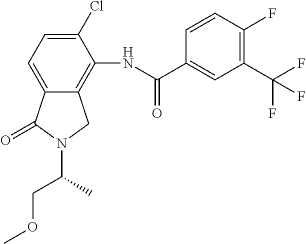

A solution of R-alaminol (4.4 g, 58.6 mmol), phthalic anhydride (8.7 g, 58.6 mmol) and TEA (2.73 mL, 20 mmol) in 100 mL of toluene was heated at reflux with azeotropic removal of H₂O with a Dean-Stark trap for 20 h. The solution was concentrated under reduced pressure. To the resulting residue was added 5 mL ether. When crystallization was complete, the solid was collected, washed with 5 mL hexanes and dried under vacuum to afford 2-((R)-2-hydroxy-1-methyl-ethyl)isoindole-1,3-dione (10.96 g, 91%).

To a stirred suspension of sodium hydride (400 mg of 60% oil suspension, 10 mmol) in mL of DMF under a nitrogen atmosphere was added 2-((R)-2-hydroxy-1-methyl-ethyl)isoindole-1,3-dione (2.0 g, 9.7 mmol). When gas evolution had ceased (5 minutes), iodomethane (1 mL, 16 mmol) was added. 1 mL DMF was added and the mixture was stirred at 50° C. for 1 h. The mixture was diluted with 15 mL H₂O and extracted with 25 mL ether. The organic phase was dried over MgSO₄ and concentrated under reduced pressure. Purification on an AnaLogix Intelliflash system (60 g column, SF25 silica, 5-15% ethyl acetate/hexanes) afforded 2-((R)-2-methoxy-1-methyl-ethyl)isoindole-1,3-dione (1.12 g, 53%): LC/MS-ESI observed [M+H]⁺ 220.

To a solution of 2-((R)-2-methoxy-1-methyl-ethyl)isoindole-1,3-dione (1.04 g, 4.7 mmol) in 5 mL ethanol was added 98% hydrazine (238 mg, 4.7 mmol) and the reaction mixture was heated at reflux for 30 minutes and allowed to cool to room temperature. The precipitate was filtered off. The filtrate was made acidic with 1.0 M HCl in ether. The solvent was removed under reduced pressure. Ethanol was added (2 mL). The solid was filtered off and the filtrate was concentrated under reduced pressure to afford (R)-2-methoxy-1-methyl-ethylamine hydrochloride (600 mg, 100%) as a red semi-solid.

To a mixture of (R)-2-methoxy-1-methyl-ethylamine hydrochloride (500 mg, 4 mmol) and K₂CO₃ (1 g, 7.2 mmol) in 1 mL H₂O and 5 mL acetonitrile was added 2-bromomethyl-3-nitro-benzoic acid methyl ester (prepared in example 19, 960 mg, 3.5 mmol) and the mixture was heated at reflux for 1 h. The mixture was diluted with 10 mL H₂O and extracted with 25 mL of ethyl acetate. The organic phase was dried over MgSO₄ and concentrated under reduced pressure. The residue was recrystallized from ether/hexanes to afford product (451 mg). Purification of the mother liquor on an AnaLogix Intelliflash system (12 g column, SF15 silica, 5-20% ethyl acetate/hexanes) afforded an additional 180 mg of product. Combined with recrystallized product to afford 2-((R)-2-methoxy-1-methylethyl)-4-nitro-2,3-dihydro-isoindol-1-one (631 mg, 72%): LC/MS-ESI observed [M+H]⁺ 251.

A mixture of 2-((R)-2-methoxy-1-methylethyl)-4-nitro-2,3-dihydro-isoindol-1-one (590 mg, 2.36 mmol) and 150 mg 10% Pd/C in 20 mL of methanol was shaken under 50 psi of hydrogen at room temperature for 30 minutes. The catalyst was removed by filtration through a Whatman GF/B filter and celite. The filtrate was concentrated under reduced pressure to afford 4-amino-2-((R)-2-methoxy-1-methyl-ethyl)-2,3-dihydro-isoindol-1-one (480 mg, 92%) as a solid.

To a solution of 4-amino-2-((R)-2-methoxy-1-methyl-ethyl)-2,3-dihydro-isoindol-1-one (460 mg, 2.09 mmol) in 10 mL of acetonitrile at 70° C. was added NCS (300 mg, 2.25 mmol) in portions and the reaction mixture was heated at 70-75° C. for 1 h. An additional 50 mg (0.37 mmol) of NCS was added. After a further 30 minutes at 75° C., the mixture was concentrated under reduced pressure. LCMS showed a mixture of 47% 7-chloro-product, 32% of the desired 5-chloro-product and 20% of the 5,7-dichloro-product. Purification on an AnaLogix Intelliflash system (60 g SF25 silica, 50:50:0.5 hexanes:ethyl acetate: acetic acid) afforded 4-amino-5-chloro-2-((R)-2-methoxy-1-methyl-ethyl)-2,3-dihydro-isoindol-1-one (130 mg, 24%).

The mixture of 4-amino-5-chloro-2-((R)-2-methoxy-1-methyl-ethyl)-2,3-dihydro-isoindol-1-one (130 mg, 0.051 mmol) and NaHCO₃ (150 mg, 1.79 mmol) and 4-fluoro-3-trifluoromethyl-phenyl)acetyl chloride (prepared in example 17, 170 mg, 0.7 mmol) and 35 mg dodecyl trimethyl ammonium bromide in 8 mL of THF was heated at reflux for 1 h. The mixture was diluted with 10 mL of H₂O, 5 mL saturated NaHCO₃ and 5 mL saturated NaCl and extracted with 20 mL ethyl acetate. The organic phase was dried over MgSO₄ and concentrated under reduced pressure. Purification on an AnaLogix Intelliflash system (24 g column, SF15 silica, 50-80% hexanes/ethyl acetate gradient) followed by recrystallization from ether/hexanes afforded N-[5-chloro-2-((R)-2-methoxy-1-methyl-ethyl)-1-oxo-2,3-dihydro-1H-isoindol-4-yl]-4-fluoro-3-trifluoromethyl-benzamide (139 mg): m.p. 131-132° C., LC/MS-ESI observed [M+H]⁺ 459.

### Biological Examples

### Intracellular Calcium Flux (FLIPR) Assay

### Compound and Reagent Preparation:

Stock solutions of compounds were prepared from powders as a 10 mM DMSO stock solution. These solutions were stored at RT during the two week period of these experiments to prevent freeze-thaw of the DMSO stocks. The DMSO stocks were added to the appropriate assay buffer at a concentration of 10 μM, and then diluted serially to the final concentrations that were tested. No observable precipitate was formed at any time during this process. The aqueous solutions of compounds as well as ATP (Sigma A7699) and BzATP (Sigma B6396) were prepared fresh for each day of experiment.

Cell culture: 1321N1-hP2X₇ and HEK293-rP2X₇

1321N1 cells stably expressing the full length human P2X₇ gene (1321N1-hP2X₇) and HEK293 cells stably expressing the full length rat P2X₇ gene (HEK293-rP2X₇) were obtained from the Roche Cell Culture Facility. 1321N1-hP2X₇ cells were grown in Dulbecco's Modified Eagle's Medium (DMEM) high glucose supplemented with 10% FBS and 250 μg/mL G418. HEK293-rP2X₇ cells were grown in DMEM/F-12 supplemented with 10% FBS, 1 mM CaCl₂, 2 mM MgCl₂, 2 mM L-Glutamine and 500 μg/ml G418. Cells were split such that they never became >70% confluent.

### Intracellular Calcium Flux (FLIPR):

On the day prior to the experiment, 1321N1-hP2X₇ or HEK293-rP2X₇ cells were released into suspension with calcium-free PBS+Versene and washed by centrifugation with calcium-free PBS to remove the Versene. Cells were resuspended in growth medium at a density of 2.5×10⁵ cells/mL and seeded into black walled, clear bottom 96 well plates (50,000 cells/well) approximately 18 hr prior to intracellular calcium flux experiments.

On the day of the experiment, plates were washed with FLIPR buffer (calcium- and magnesium-free Hank's Balanced Salt Solution (HBSS) supplemented with 10 mM Hepes, 2.5 mM probenecid and 2 mM calcium chloride) using a BIO-TEK 96 channel plate washer and incubated with 2 mM fluo-3 dye at 37° C. for one hr. The dye was then removed by plate washing and the cells were allowed to equilibrate for 20 min at room temperature with antagonist or vehicle (FLIPR buffer). Agonist (100 μM BzATP final concentration for hP2X₇; 5 μM BzATP final concentration or rP2X₇) was added online with the FLIPR and fluorescence measurements made at 1 sec intervals for 60 sec followed by 3 sec intervals for a further 4 min (5 min total). A final addition of 5 μM ionomycin was made and the maximal BzATP-evoked fluorescence normalized to the maximal ionomycin-evoked fluorescence.

| Compound | FLIPR pIC50 hP2X7-1321n1 |
|---|---|
| I-1a | 6.81 |
| I-1b | 7.26 |
| I-2 | 6.13 |
| I-3 | 6.97 |
| I-4 | 6.14 |
| I-5 | 6.45 |
| I-6 | 6.38 |
| I-7 | 6.07 |
| I-8 | 6.36 |
| I-9 | 6.02 |
| I-10 | 6.74 |
| I-11 | 7.00 |
| I-12 | 7.51 |
| I-13 | 6.96 |
| I-14 | 6.04 |
| I-15 | 6.15 |
| I-16 | 6.69 |
| I-17 | 6.76 |
| I-18a | 7.40 |
| I-18b | 6.27 |
| I-19 | 6.48 |
| I-20 | 6.05 |
| I-21 | 6.41 |

### Human Whole Blood IL-1β Release Assay

### Compound & Reagent Preparation:

10 mM stock solutions of compounds in DMSO (Sigma D2650) were prepared and used either fresh or after storage at −20° C. Appropriate (200×) serial dilutions of the compounds were made in DMSO, then freshly diluted 1 to 20 (10×) with Dulbecco's phosphate buffered saline (DPBS; Mediatech Inc., 21-030), such that final DMSO concentration in the blood always equaled 0.5%.

30 mM ATP (Sigma A7699) was prepared immediately before use in 50 mM HEPES (Gibco 15630) and the pH adjusted to 7.2 with 1M sodium hydroxide.

### Blood Donors:

Human blood donors were medication free and restricted from utilizing alcohol or caffeine for at least the 24 hr preceding collection. The blood was collected into sodium heparin vacutainer tubes and used the same day.

### Assay Method:

The OptEIA Human IL-1β ELISA Set, OptEIA Coating Buffer, Assay Diluent and TMB Substrate Reagent Set used in the assay were commercially obtained from BD Pharmingen. Blood was diluted 1:1 with Dulbecco's PBS, LPS (Escherichia Coli 0127:B8, Sigma L3129) added to a final concentration of 25 ng/mL and incubated for 2 hr at 37° C. 48 μL of this LPS primed blood was added to 6 μL of the 10× compound in 5% DMSO/PBS in the appropriate well of a 96-well polypropylene plate. The blood and compound were mixed and allowed to incubate for 30 min at 37° C. 6 μl of 30 mM ATP was added to the LPS-primed blood+compound, mixed thoroughly and incubated for a further 30 min at 37° C. 96 μL of ELISA assay buffer was added to each well and the plate centrifuged at 4° C. 1,200 rpm for 10 min. Supernatant was removed and assayed for IL-1β using the OptiEIA kit according to the manufacturer's protocol (Serum may be frozen at −2° C. prior to assay). IC₅₀s were calculated using XLfit.

### In Vivo Assay for Asthma and Lung Function:

BALb/cJ mice are immunized with a standard immunization protocol. Briefly, mice (N=8/group) are immunized i.p. with ovalbumin (OVA; 10 μg) in alum on days 0 and 14. Mice are then challenged with aerosolized OVA (5%) on day 21 and 22. Animals receive vehicle (p.o.) or a compound of the invention (100 mg/kg p.o.) all starting on day 20.

Lung function is evaluated on day 23 using the Buxco system to measure PenH in response to an aerosol methacholine challenge. Mice are then euthanized and plasma samples collected at the end of the study.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective spirit and scope of the present invention. All such modifications are intended to be within the scope of the claims appended hereto.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

We claim:

1. A compound of formula I: 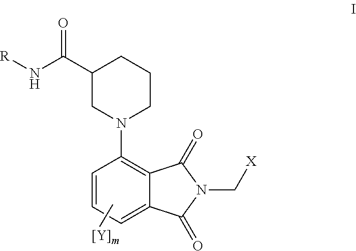
wherein:
R is R′ or CH₂R′;
R′ is aryl, cycloalkyl, heterocycloalkyl, heteroaryl, optionally substituted with one or more R¹;
R¹ is halo, hydroxy, lower alkyl, lower haloalkyl, lower hydroxyalkyl, lower alkoxy, lower thioalkyl, lower alkyl sulfonyl, or lower haloalkoxy;
X is lower alkyl, aryl, cycloalkyl, heterocycloalkyl, or heteroaryl, optionally substituted with one or more X¹;
X¹ is halo, hydroxy, lower alkyl, lower haloalkyl, lower hydroxyalkyl, or lower alkoxy;
Y is halo;
m is 0, 1, or 2;
or a pharmaceutically acceptable salt thereof.

2. The compound of claim 1, wherein X is pyridyl, optionally substituted with one or more X¹.

3. The compound of claim 2, wherein R is phenyl, optionally substituted with one or more R¹.

4. The compound of claim 3, wherein m is 0.

5. The compound of claim 3, wherein m is 1.

6. The compound of claim 5, wherein Y is Cl.

7. The compound of claim 3, wherein m is 2.

8. The compound of claim 7, wherein each Y is Cl.

9. The compound of claim 3, wherein R¹ is halo.

10. The compound of claim 3, wherein R¹ is alkoxy.

11. The compound of claim 3, wherein R¹ is haloalkyl.

12. The compound of claim 1, wherein R is heterocycloalkyl, optionally substituted with one or more R¹.

13. The compound of claim 1, wherein R is heteroaryl, optionally substituted with one or more R¹.

14. The compound of claim 1, wherein R is cycloalkyl, optionally substituted with one or more R¹.

15. The compound of claim 1, wherein X is heteroaryl, optionally substituted with one or more X¹.

16. The compound of claim 1, wherein X is lower alkyl, optionally substituted with one or more X¹.

17. A compound of formula II: 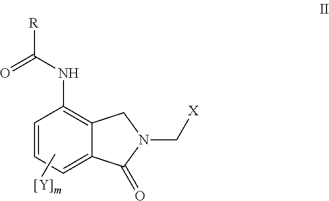
wherein:
R is R′ or CH₂R′;
R′ is aryl or cycloalkyl, optionally substituted with one or more R¹;
R¹ is halo, hydroxy, lower alkyl, lower haloalkyl, lower hydroxyalkyl, lower alkoxy, lower thioalkyl, lower alkyl sulfonyl, or lower haloalkoxy;
X is lower alkyl, optionally substituted with one or more X¹;
X¹ is halo, hydroxy, lower alkyl, lower haloalkyl, lower hydroxyalkyl, or lower alkoxy;
Y is halo;
m is 0, 1, or 2;
or a pharmaceutically acceptable salt thereof.

18. A compound selected from the group consisting of:
1-(1,3-Dioxo-2-pyridin-4-ylmethyl-2,3-dihydro-1H-isoindol-4-yl)-piperidine-3-carboxylic acid 2-methoxy-benzylamide;
(R)-1-(1,3-Dioxo-2-pyridin-4-ylmethyl-2,3-dihydro-1H-isoindol-4-yl)-piperidine-3-carboxylic acid 2-methoxy-benzylamide;
(S)-1-(1,3-Dioxo-2-pyridin-4-ylmethyl-2,3-dihydro-1H-isoindol-4-yl)-piperidine-3-carboxylic acid 2-methoxy-benzylamide;
(R)-1-(1,3-Dioxo-2-pyridin-4-ylmethyl-2,3-dihydro-1H-isoindol-4-yl)-piperidine-3-carboxylic acid (adamantan-1-ylmethyl)-amide;
(R)-1-(1,3-Dioxo-2-pyridin-4-ylmethyl-2,3-dihydro-1H-isoindol-4-yl)-piperidine-3-carboxylic acid (2-methoxy-phenyl)-amide;
(R)-1-[1,3-Dioxo-2-(tetrahydro-pyran-4-ylmethyl)-2,3-dihydro-1H-isoindol-4-yl]-piperidine-3-carboxylic acid 2-methoxy-benzylamide;
(R)-1-(1,3-Dioxo-2-thiazol-5-ylmethyl-2,3-dihydro-1H-isoindol-4-yl)-piperidine-3-carboxylic acid 2-methoxy-benzylamide;
(R)-1-(1,3-Dioxo-2-pyridin-4-ylmethyl-2,3-dihydro-1H-isoindol-4-yl)-piperidine-3-carboxylic acid 2-trifluoromethoxy-benzylamide;
(R)-1-(1,3-Dioxo-2-pyridin-4-ylmethyl-2,3-dihydro-1H-isoindol-4-yl)-piperidine-3-carboxylic acid (thiophen-2-ylmethyl)-amide;
(R)-1-(1,3-Dioxo-2-pyridin-4-ylmethyl-2,3-dihydro-1H-isoindol-4-yl)-piperidine-3-carboxylic acid 2-ethoxy-benzylamide;
(R)-1-(1,3-Dioxo-2-pyridin-4-ylmethyl-2,3-dihydro-1H-isoindol-4-yl)-piperidine-3-carboxylic acid 2,3-dimethoxy-benzylamide;
(R)-1-(1,3-Dioxo-2-pyridin-4-ylmethyl-2,3-dihydro-1H-isoindol-4-yl)-piperidine-3-carboxylic acid 2-hydroxy-benzylamide;
(R)-1-(1,3-Dioxo-2-pyridin-4-ylmethyl-2,3-dihydro-1H-isoindol-4-yl)-piperidine-3-carboxylic acid 2-methylsulfanyl-benzylamide;
(R)-1-(1,3-Dioxo-2-pyridin-4-ylmethyl-2,3-dihydro-1H-isoindol-4-yl)-piperidine-3-carboxylic acid 2-fluoro-benzylamide;
(R)-1-(1,3-Dioxo-2-pyridin-4-ylmethyl-2,3-dihydro-1H-isoindol-4-yl)-piperidine-3-carboxylic acid ([1,4]dioxan-2-ylmethyl)-amide;
(R)-1-(1,3-Dioxo-2-pyridin-4-ylmethyl-2,3-dihydro-1H-isoindol-4-yl)-piperidine-3-carboxylic acid cyclohexylmethyl-amide;
(R)-1-(1,3-Dioxo-2-pyridin-4-ylmethyl-2,3-dihydro-1H-isoindol-4-yl)-piperidine-3-carboxylic acid 2-methanesulfonyl-benzylamide;
N-[5-Chloro-2-((R)-2-hydroxy-1-methyl-ethyl)-1-oxo-2,3-dihydro-1H-isoindol-4-yl]-2-(4-fluoro-3-trifluoromethyl-phenyl)-acetamide;
2-Adamantan-1-yl-N-[5-chloro-2-((R)-2-hydroxy-1-methyl-ethyl)-1-oxo-2,3-dihydro-1H-isoindol-4-yl]-acetamide;
2-Adamantan-1-yl-N-[5,7-dichloro-2-((R)-2-hydroxy-1-methyl-ethyl)-1-oxo-2,3-dihydro-1H-isoindol-4-yl]-acetamide;
2-(4-Fluoro-3-trifluoromethyl-phenyl)-N-[2-(2-methoxy-1-methyl-ethyl)-1-oxo-2,3-dihydro-1H-isoindol-4-yl]-acetamide;
N-[5-Chloro-2-(2-methoxy-ethyl)-1-oxo-2,3-dihydro-1H-isoindol-4-yl]-2-(4-fluoro-3-trifluoromethyl-phenyl)-acetamide; and
N-[5-Chloro-2-((R)-2-methoxy-1-methyl-ethyl)-1-oxo-2,3-dihydro-1H-isoindol-4-yl]-2-(4-fluoro-3-trifluoromethyl-phenyl)-acetamide.

19. A pharmaceutical composition comprising:
(a) a pharmaceutically acceptable carrier; and
(b) a compound of claim 18.

20. A method for treating arthritis, said method comprising administering to a subject in need thereof a therapeutically effective amount of a compound of claim 18.

21. A method for treating a pain condition selected from inflammatory pain, surgical pain, visceral pain, dental pain, premenstrual pain, central pain, pain due to burns, migraine or cluster headaches, nerve injury, neuritis, neuralgias, poisoning, ischemic injury, interstitial cystitis, cancer pain, viral, parasitic or bacterial infection, post-traumatic injury, or pain associated with irritable bowel syndrome, said method comprising administering to a subject in need thereof a therapeutically effective amount of a compound of claim 18.

22. A method for treating a respiratory disorder selected from chronic obstructive pulmonary disorder (COPD), asthma, and bronchospasm, said method comprising administering to a subject in need thereof a therapeutically effective amount of a compound of claim 18.

23. A method for treating diabetes, said method comprising administering to a subject in need thereof a therapeutically effective amount of a compound of claim 18.
